SEQUENCE LISTING (51 sequences)

SEQ ID NO: 1 - DNA, 59 bp, Artificial Sequence
  Primer
attctagaggccgaggcggccgacatgttttttttttttttttttttttttttttttvn

SEQ ID NO: 2 - DNA, 39 bp, Artificial Sequence
  Primer
aagcagtggtatcaacgcagactggccattacggccggg

SEQ ID NO: 3 - DNA, 18 bp, Artificial Sequence
  Hybridization Tag
aaaacatccttccaccga

SEQ ID NO: 4 - DNA, 18 bp, Artificial Sequence
  Hybridization Tag
gtccttctgtccgctcaa

SEQ ID NO: 5 - DNA, 18 bp, Artificial Sequence
  Hybridization Tag
ggcggaatgagatacgat

SEQ ID NO: 6 - DNA, 18 bp, Artificial Sequence
  Hybridization Tag
tcgcactttttcgcataa

SEQ ID NO: 7 - DNA, 18 bp, Artificial Sequence
  Hybridization Tag
accgactggaaccgaata

SEQ ID NO: 8 - DNA, 18 bp, Artificial Sequence
  Hybridization Tag
gcaaaacaatggcgagta

SEQ ID NO: 9 - DNA, 18 bp, Artificial Sequence
  Hybridization Tag
tggtctggtctggtctgg

SEQ ID NO: 10 - DNA, 18 bp, Artificial Sequence
  Hybridization Tag
gaaaagcaaaccaaaccc

SEQ ID NO: 11 - DNA, 18 bp, Artificial Sequence
  Hybridization Tag
accctcctctccacgatt

SEQ ID NO: 12 - DNA, 18 bp, Artificial Sequence
  Hybridization Tag
aaggggatgggaaagtct

SEQ ID NO: 13 - DNA, 18 bp, Artificial Sequence
  Hybridization Tag
ttcctcttcctcttgcca

SEQ ID NO: 14 - DNA, 18 bp, Artificial Sequence
  Hybridization Tag
tgcggctggacttactct

SEQ ID NO: 15 - DNA, 18 bp, Artificial Sequence
  Hybridization Tag
agccacagcccagtttag

SEQ ID NO: 16 - DNA, 18 bp, Artificial Sequence
  Hybridization Tag
attgaagcccgaacagac

SEQ ID NO: 17 - DNA, 18 bp, Artificial Sequence
  Hybridization Tag
ggctgcgttcaatcatct

SEQ ID NO: 18 - DNA, 18 bp, Artificial Sequence
  Hybridization Tag
ataccaaaaagcgagcct

SEQ ID NO: 19 - DNA, 18 bp, Artificial Sequence
  Hybridization Tag
tactaacgcccctggtct

SEQ ID NO: 20 - DNA, 18 bp, Artificial Sequence
  Hybridization Tag
gcccctgactcttgctaa

SEQ ID NO: 21 - DNA, 18 bp, Artificial Sequence
  Hybridization Tag
cttggtcggtcctttttg

SEQ ID NO: 22 - DNA, 18 bp, Artificial Sequence
  Hybridization Tag
agcggtgagtggagaaaa

SEQ ID NO: 23 - DNA, 18 bp, Artificial Sequence
  Hybridization Tag
tcgtcgtttgggtctctt

SEQ ID NO: 24 - DNA, 18 bp, Artificial Sequence
  Hybridization Tag
gtggtggggttgtgagaa

SEQ ID NO: 25 - DNA, 18 bp, Artificial Sequence
  Hybridization Tag
aaacgaaacggaaccact

SEQ ID NO: 26 - DNA, 18 bp, Artificial Sequence
  Hybridization Tag
ccacgcacaaaaagaatc

SEQ ID NO: 27 - DNA, 18 bp, Artificial Sequence
  Hybridization Tag
tttggtttgggcttgtct

SEQ ID NO: 28 - DNA, 18 bp, Artificial Sequence
  Hybridization Tag
cgatgttgcccctactgt

SEQ ID NO: 29 - DNA, 18 bp, Artificial Sequence
  Hybridization Tag
ttcgctgtggctctgtta

SEQ ID NO: 30 - DNA, 18 bp, Artificial Sequence
  Hybridization Tag
tcagttttccgcatttca

SEQ ID NO: 31 - DNA, 18 bp, Artificial Sequence
  Hybridization Tag
tattcaaaacgggaggct

SEQ ID NO: 32 - DNA, 18 bp, Artificial Sequence
  Hybridization Tag
ttgggtggcagataggtc

SEQ ID NO: 33 - DNA, 18 bp, Artificial Sequence
  Hybridization Tag
ttgtttttgggggtaggt

SEQ ID NO: 34 - DNA, 18 bp, Artificial Sequence
  Hybridization Tag
agggtggaaaatgcgata

SEQ ID NO: 35 - DNA, 18 bp, Artificial Sequence
  Hybridization Tag
agagtggcgagtgtaggg

SEQ ID NO: 36 - DNA, 18 bp, Artificial Sequence
  Hybridization Tag
ataaggacccagccacaa

SEQ ID NO: 37 - DNA, 18 bp, Artificial Sequence
  Hybridization Tag
atcggctggcaataagtc

SEQ ID NO: 38 - DNA, 18 bp, Artificial Sequence
  Hybridization Tag
aggcaagtggagcagtgt

SEQ ID NO: 39 - DNA, 18 bp, Artificial Sequence
  Hybridization Tag
agagaaacggcacccata

SEQ ID NO: 40 - DNA, 18 bp, Artificial Sequence
  Hybridization Tag
tccttcttggtctcgctt

SEQ ID NO: 41 - DNA, 18 bp, Artificial Sequence
  Hybridization Tag
aggaaaaagccatcgtca

SEQ ID NO: 42 - DNA, 18 bp, Artificial Sequence
  Hybridization Tag
acggagaatggcgagata

SEQ ID NO: 43 - DNA, 18 bp, Artificial Sequence
  Hybridization Tag
tgaccttgctgacccttt

SEQ ID NO: 44 - DNA, 18 bp, Artificial Sequence
  Hybridization Tag
tgttgtgcgtgttggaag

SEQ ID NO: 45 - DNA, 18 bp, Artificial Sequence
  Hybridization Tag
gtttttgtgcctttcggt

SEQ ID NO: 46 - DNA, 18 bp, Artificial Sequence
  Hybridization Tag
gagtttctggagcggttg

SEQ ID NO: 47 - DNA, 22 bp, Artificial Sequence
  Primer
ggattgcccaatacttaacact

SEQ ID NO: 48 - DNA, 20 bp, Artificial Sequence
  Primer
acaagcctgctttggtgtgt

SEQ ID NO: 49 - DNA, 19 bp, Artificial Sequence
  Primer
ctggagcgtggacaatatg

SEQ ID NO: 50 - DNA, 20 bp, Artificial Sequence
  Primer
caagaaccctttcctcttcc

SEQ ID NO: 51 - DNA, 12 bp, Artificial Sequence
  Linker
cccccccccccc

(19) United States
(12) Patent Application Publication
(10) Pub. No.: US 2003/0165865 A1
(43) Pub. Date: Sep. 4, 2003

(54) Methods of analysis of nucleic acids

(76) Inventors: Christopher A. Hinkel, San Diego, CA (US); William J. Kimmerly, Agoura Hills, CA (US); Li Yang, San Diego, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 10/056,908

(22) Filed: Jan. 25, 2002

Related U.S. Application Data
(60) Provisional application No. 60/264,972, filed on Jan. 29, 2001.
(60) Provisional application No. 60/266,186, filed on Feb. 2, 2001.
(60) Provisional application No. 60/295,986, filed on Jun. 4, 2001.

(51) Int. Cl. C12Q 1/68; C12P 19/34

(52) U.S. Cl. 435/6; 435/91.2

(57) ABSTRACT

Methods are provided for the multiplex analysis of polynucleotide expression and single nucleotide polymorphism detection using capture probes coupled to uniquely identified particles. The methods provided are characterized by high flexibility and high throughput.

### CROSS REFERENCE TO RELATED APPLICATIONS

This application claims the benefit of U.S. Provisional Patent Application Serial No. 60/264,972, filed Jan. 29, 2001; U.S. Provisional Patent Application Serial No. 60/266,186, filed Feb. 2, 2001; and U.S. Provisional Patent Application No. 60/295,986, filed Jun. 4, 2001; each of which is herein incorporated by reference in its entirety for all purposes.

### BACKGROUND

The field of genomics has taken rapid strides in recent years. It started with efforts to determine the entire nucleotide sequence of simpler organisms such as viruses and bacteria. As a result, genomic sequences of Hemophilus influenzae (Fleischman et al., Science 269: 496-512, 1995) and a number of other bacterial strains (Escherichia coli, Mycobacterium tuberculosis, Helicobacter pylori, Caulobacter jejuni, Mycobacterium leprae) are now available (reviewed in Nierman et al., Curr. Opin. Struct. Biol 10: 343-348, 2000). This was followed by the determination of complete nucleotide sequence of a number of eukaryotic organisms including budding-yeast (Saccharomyces cerevisiae) (Goffeau et al., Science 274: 563-567, 1996), nematode (Cenorhabditis elegans) (C. elegans sequencing consortium, Science 282: 2012-2018 1998) and fruit fly (Drosophila melanogaster) (Adams et al., Science 287: 2185-2195, 2000). The sequence of the human genome was published in February of 2001 (International Human Genome Sequence Consortium, Nature, 409:860-921, 2001; Venter et al., Science, 291:1304-1351, 2001). Additionally, some of the ongoing efforts are currently focused on genome sequencing of agriculturally important plants such as rice (Science 288: 239-240, 2000; Sasaki and Burr, Curr. Opin. Plant Biol. 3: 138-141, 2000) and experimentally critical animal model such as mice (News Focus, Science 288: 248-257, 2000).

The availability of complete genomic sequences of various organisms promises to significantly advance our understanding of various fundamental aspects of biology. It also promises to provide unparalleled applied benefits such as understanding genetic basis of certain diseases, providing new targets for therapeutic intervention, developing a new generation of diagnostic tests, etc. New and improved tools, however, will be needed to harvest and fully realize the potential of genomics research.

Even though the DNA complement or gene complement is identical in various cells in the body of multi-cellular organisms, there are qualitative and quantitative differences in gene expression in various cells. A human genome is estimated to contain roughly about 30,000-40,000 genes, however, only a fraction of these genes are expressed in a given cell (International Human Genome Sequence Consortium, Nature, 409:860-921, 2001; Venter et al., Science, 291:1304-1351, 2001). Moreover, there are quantitative differences among the expressed genes in various cell types. Although all cells express certain housekeeping genes, each distinct cell type additionally expresses a unique set of genes. Phenotypic differences between cell types are largely determined by the complement of proteins that are uniquely expressed. It is the expression of this unique set of genes and the encoded proteins, which constitutes functional identity of a cell type, and distinguishes it from other cell types. Moreover, the complement of genes that are expressed, and their level of expression vary considerably depending on the developmental stage of a given cell type. Certain genes are specifically activated or repressed during differentiation of a cell. The level of expression also changes during development and differentiation. Qualitative and quantitative changes in gene expression also take place during cell division, e.g. in various phases of cell cycle. Signal transduction by biologically active molecules such as hormones, growth factors and cytokines often involves modulation of gene expression. Global change in gene expression also plays a determinative role in the process of aging.

In addition to the endogenous or internal factors as mentioned above, certain external factors or stimuli, such as environmental factors, also bring about changes in gene expression profile. Infectious organisms such as bacteria, viruses, fungi and parasites interact with the cells and influence the qualitative and quantitative aspects of gene expression. Thus, precise complement of genes expressed by a given cell type is influenced by a number of endogenous and exogenous factors. The outcome of these changes is critical for normal cell survival, growth, development and response to environment. Therefore, it is important to identify, characterize and measure changes in gene expression. The knowledge gained from such analysis will not only further our understanding of basic biology, but it will also allow us to exploit it for various purposes such as diagnosis of infectious and non-infectious diseases, screening to identify and develop new drugs, etc.

Besides the conventional, one by one gene expression analysis methods like Northern analysis, RNase protection assays, and real time PCT (RT-PCR); there are several methods currently available to examine gene expression in a genome wide scale. These approaches are variously referred to as RNA profiling, differential display, etc. These methods can be broadly divided into three categories: (1) hybridization-based methods such as subtractive hybridization (Koyama et al., Proc. Natl. Acad. Sci. USA 84: 1609-1613, 1987; Zipfel et al., Mol. Cell. Biol. 9: 1041-1048, 1989), microarray (U.S. Pat. No. 6,150,095), etc., (2) cDNA tags: EST, serial analysis of gene expression (SAGE) (see, e.g. U.S. Pat. Nos. 5,695,937 and 5,866,330), and (3) fragment size based, often referred to as gel-based methods where a differential display is generated upon electrophoretic separation of DNA fragments on a gel such as a polyacrylamide gel (described in U.S. Pat. Nos. 5,871,697, 5,459,037, 5,712,126 and PCT publication No. WO 98/51789).

Microarray based gene analysis approach enables working with hundreds of thousands of genes simultaneously rather than one or a few genes at a time. Microarray technology has come at an appropriate time, when entire genomes of humans and other organisms are being worked out. Massive sequence information generated as a result of genome sequencing, particularly human genome sequencing, has created a demand for technologies that provide high-throughput and speed. Microarrays fill this unique niche. Most of the complex physiological processes precede or succeed change in the expression of a large number of genes. Techniques that were available before the advent of microarrays are not suitable to monitor such large-scale changes in gene expression. DNA microarrays offer the opportunity to perform fast, comprehensive, moderately quantitative analyses on hundreds of thousands of genes simultaneously. A DNA microarray is composed of an ordered set of DNA molecules of known sequences usually arranged in rectangular configuration in a small space such as 1 cm² in a standard microscope slide format. For example, an array of 200×200 would contain 40,000 spots with each spot corresponding to a probe of known sequence. Such a microarray can be potentially used to simultaneously monitor the expression of 40,000 genes in a given cell type under various conditions. The probes usually take the form of cDNA, ESTs or oligonucleotides. Most preferred are ESTs and oligonucleotides in the range of 30-200 bases long as they provide an ideal substrate for hybridization. There are two approaches to building these microarrays, also known as chips, one involving covalent attachment of pre-synthesized probes, the other involving building or synthesizing probes directly on the chip. The sample or test material usually consists of RNA that has been amplified by PCR. PCR serves the dual purposes of amplifying the starting material as well as allowing introduction of fluorescent tags. For a detailed discussion of microarray technology, see e.g., Graves, Trends Biotechnol. 17: 127-134, 1999.

High-density microarrays are built by depositing an extremely minute quantity of DNA solutions at precise location on an array using high precision machines, a number of which are available commercially. An alternative approach pioneered by Packard Instruments, enables deposition of DNA in much the same way that ink jet printer deposits spots on paper. High-density DNA microarrays are commercially available from a number of sources such as Affymetrix, Incyte, Mergen, Genemed Molecular Biochemicals, Sequenom, Genomic Solutions, Clontech, Research Genetics, Operon and Stratagene. Currently, labeling for DNA microarray analysis involves fluorescence, which allows multiple independent signals to be read at the same time. This allows simultaneous hybridization of the same chip with two samples labeled with different fluorescent dyes. The calculation of the ratio of fluorescence at each spot allows determination of the relative change in the expression of each gene under two different conditions. For example, comparison between a normal tissue and a corresponding tumor tissue using the approach helps in identifying genes whose expression is significantly altered. Thus, the method offers a particularly powerful tool when the gene expression profile of the same cell is to be compared under two or more conditions. High-resolution scanners with capability to monitor fluorescence at various wavelengths are commercially available.

As greater information on the genome of species is obtained, new markers in the form of genetic variations or polymorphisms have been identified for various traits. Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequences repeats (SSR) or microsatellites. These repeats can be dinucleotide, trinucleotide, tetranucleotide or pentanucleotide repeats. Polymorphism results from variation in the number of repeated sequences found at a particular locus.

Recently, attention has focused on single nucleotide polymorphisms (SNPs), which are by far the most common source of variation in the genome, as useful genetic markers. SNPs account for approximately 90% of human DNA polymorphism (Collins et al., Genome Res., 8:1229-1231, 1998). SNPs are single base pair positions in genomic DNA at which different sequence alternatives (alleles) exist in a population. The term SNP is not limited to single base substitutions, but also includes single base insertions or deletions. In addition, short insertions or deletions of 10 base pairs or less are also often categorized as SNPs because they are often detected with methodologies used to detect single base polymorphisms.

Nucleotide substitution SNPs are of two types. A transition is the replacement of one purine by another purine or one pyrimidine by another pyrimidine. A transversion is the replacement of a purine for a pyrimidine or vice versa. The typical frequency at which SNPs are observed is about 1 per 1000 base pairs (Li and Sadler, Genetics, 129:513-523, 1991; Wang et al., Science 280:1077-1082, 1998; Harding et al., Am. J. Human Genet., 60:772-789, 1997; Taillon-Miller et al., Genome Res., 8:748-754, 1998). The frequency of SNPs varies with the type and location of the change in question. In base substitutions, two-thirds of the substitutions involve the C⇄T (G⇄A) type. This variation in frequency is thought to be related to 5-methylcytosine deamination reactions that occur frequently, particularly at CpG dinucleotides. In regard to location, SNPs occur at a much higher frequence in non-coding regions than they do in coding regions.

There are various ways in which SNPs can affect phenotype. Studies have shown that SNPs can cause major changes in structural folds of mRNA that may affect cell regulation (Shen et al., Proc. Natl. Acad. Sci. USA, 96:7871-7876, 1999). When located in a coding region, the presence of a SNP can result in the production of a protein that is non-functional or has decreased function. When present in a non-coding regulatory region, such as a promoter region, the SNP can alter expression of a gene.

Several methods for the detection of SNPs are known in the art. These include multiplexed allele-specific diagnostic assay (MASDA; U.S. Pat. No. 5,834,181), TaqMan assay (U.S. Pat. No. 5,962,233), molecular beacons (U.S. Pat. No. 5,925,517), microtiter array diagonal gel electrophoresis (MADGE, Day and Humphries, Anal. Biochem., 222:389-395, 1994), PCR amplification of specific alleles (PASA, Sommer et al., Mayo Clin. Proc., 64:1361-1372, 1989), allele specific amplification (A S A, Nichols, Genomics, 5:535-540, 1989), allele-specific PCR (Wu et al., Proc. Natl. Acad. Sci. USA, 86:2757-2760, 1989), amplification refractory mutation system (ARMS, Newton et al., Nuc. Acids Res., 17:2503-2516, 1989), bi-PASA (Liu et al., Genome Res., 7:389-398, 1997), ligase chain reaction (L C R, Barany, Proc. Natl. Acad. Sci. USA, 88:189-193, 1991), oligonucleotide ligation assays (OLA, U.S. Pat. No. 5,830,711; Landegren et al., Science, 214:1077-1080, 1988; Samotiaki et al., Genomics, 20:238-242, 1994; Day et al., Genomics, 29:152-162, 1995; Grossman et al., Nuc. Acids Res., 22:4527-4534, 1994), dye-labeled oligonucleotide ligation (U.S. Pat. No. 5,945,283; Chen et al., Genome Res., 8:549-556, 1998), restriction fragment length polymorphism (RFLP, U.S. Pat. Nos. 5,324,631 and 5,645,995), MALDI-TOF (Bray et al., Hum. Mutat., 17:296-304, 2001), Invader Assay (Hsu et al., Clin. Chem, 47:1373-1377, 2001) and minisequencing either alone (U.S. Pat. Nos. 5,846,710 and 5,888,819; Syvanen et al., Am. J. Hum. Genet., 52:46-59, 1993) or in combination with microarrays (Shumaker et al., Human Mut., 7:346-354, 1996) or fluorescence resonance energy transfer (U.S. Pat. No. 5,945,283; Chen et al., Proc. Natl. Acad. Sci. USA, 94:10756-10761, 1997).

As the amount of genetic information available continues to grow, the need for rapid, cost effective methods of mass gene expression and SNP analysis also grows. The wide scale application of many available methods is limited by high costs associated with consumables used, instrumentation required, the amount of labor involved, or some combination of these three factors. What is need therefore, are methods for nucleic acid analysis that allow for mass screening in a cost effective manner. The present inventive discovery meets this need.

### SUMMARY

Among the several aspects of the present inventive discovery is provided a method for determining polynucleotide expression comprising providing at least one target polynucleotide having a 3′ end and a 5′ end. The method uses a first oligonucleotide primer, at least of portion of which is capable of hybridizing to the target polynucleotide, preferably under stringent conditions, highly stringent conditions, very highly stringent conditions or extremely stringent conditions. This primer is used to obtain a first strand cDNA by reverse transcription of the polynucleotide of interest, the cDNA also having a 3′ end and a 5′ end, wherein the 5′ end of the first strand cDNA contains a sequence corresponding to the first oligonucleotide primer and the 3′ end extends at least one nucleotide beyond the 5′ end of the target polynucleotide to provide a single-stranded extension. A second oligonucleotide is also provided, at least of portion of which is capable of hybridizing to the single-stranded extension preferably under stringent conditions, highly stringent conditions, very highly stringent conditions or extremely stringent conditions, and extending the first strand of cDNA using the second oligonucleotide primer as a template to produce an extended first strand cDNA containing the first oligonucleotide primer and a region complementary to the second oligonucleotide primer. The extended first strand cDNA is then amplified, preferably in the presence of at least one detectable label, to produce amplified cDNA containing the at least one label. The amplified cDNA is digested to produce a digested cDNA and the digested cDNA hybridized to a capture probe coupled to a solid particle under stringent, conditions, preferably highly stringent conditions, very highly stringent conditions or extremely stringent conditions, where the capture probe is specific for the target polynucleotide and the particle identifies the capture probe. The identity and so the presence of the target polynucleotide of interest is determined by detecting if the digested cDNA has hybridized to the capture probe, using the particle to determine the identity of the capture probe and thus the target polynucleotide.

In one embodiment, the particle is a microbead. In another embodiment, the particle is a fluorescent particle, for example, a fluorescent microparticle or microbead. In still another embodiment, the method uses groups of particles, for example fluorescent particles, each group having a unique detectable signature, for example a fluorescent signature, and the particles of each group having a different capture probe specific for a polynucleotide of interest. As used herein, “signature” refers to a detectable marker, for example a fluorescent dye, that allows members of one group of particles to be distinguished from other groups of particles being used. In yet another embodiment, the identity and/or presence of the target polynucleotide is accomplished using a flow cytometer.

A further embodiment provides a method for diagnosing a disease condition, disorder, or predisposition in a test subject comprising determining polynucleotide expression in a test subject by the novel methods disclosed herein; determining polynucleotide expression in a reference subject known to have the disease, condition, disorder, or predisposition using the same novel method; and comparing polynucleotide expression in the test subject to polynucleotide expression in the reference subject. A related embodiment provides a method for determining the physiological or developmental state of a cell or tissue comprising, determining polynucleotide expression in a test cell or tissue by the novel methods disclosed herein; determining polynucleotide expression in a reference cell or tissue of a known physiological or developmental state by the same methods; and comparing polynucleotide expression in the test cell or tissue to polynucleotide expression in the reference cell or tissue. In one embodiment, the test subject and reference subject are animals or plants, preferably vertebrate animals or vascular plants.

Another aspect provides a method for detecting a single nucleotide polymorphism (SNP) comprising providing at least one primer pair. The primer pair contains a reverse primer and a forward primer; the forward primer having a 3′ end specific for a single nucleotide polymorphism of interest and a hybridization tag that identifies the primer. The hybridization tag is chosen so that it is not complementary to the nucleotide sequence containing the SNP of interest. The hybridization tag may be attached directly to the primer or may be coupled through a linker molecule. The primer pair is combined with a sample containing single-stranded polynucleotides under stringent conditions, preferably highly stringent conditions, very highly stringent conditions or extremely stringent conditions, which allow hybridization of the primers to complementary sequences on the single-stranded polynucleotides. The primers are extended by a primer extension reaction to produce an extension product containing the hybridization tag and a detectable label. The extension products are hybridized under stringent conditions, preferably highly stringent conditions, very highly stringent conditions or extremely stringent conditions, to a capture probe using the hybridization tag or its complement. The capture probe is, in turn, coupled to a particle, for example a microbead, where the particle serves to identify the capture probe, for example by the presence of a fluorescent dye. The hybridization of the extension product is determined by using the detectable label and the identity and/or presence of the SNP is determined based on the identity of the particle. That is, the particle identities which capture probe the hybridization tag is hybridized to, which in turn identifies the SNP, since the hybridization tag identifies the primer and therefore identifies the SNP.

In one embodiment, the reverse primer comprises a detectable label, while in another embodiment, the reverse primer is a universal reverse primer. In still another embodiment the primer extension reaction is repeat at least once, preferably multiple times such as in PCR amplification. In yet another embodiment the identity of the particle is determined on the basis of a unique fluorescent signature or tag. In a further embodiment, there are multiple primer pairs where each primer pair is specific for a different SNP, thus allowing for the detection of multiple SNPs simultaneously. In yet a further embodiment, the detection of the SNP or SNPs is by flow cytometry.

An additional aspect provides a method for detecting a single nucleotide polymorphism (SNP) comprising providing at least one polynucleotide primer having a 3′ end specific for a SNP of interest and containing a hybridization tag that serves to identify the primer. The primer is combined with a sample containing single-stranded polynucleotides under stringent conditions, preferably highly stringent conditions, very highly stringent conditions or extremely stringent conditions, which allow hybridization of the primer to complementary sequences contained in the single-stranded polynucleotides. The hybridized primers are then extended by a primer extension reaction to produce an extension product which contains the hybridization tag and a detectable label. The extension product is then hybridized to a capture probe coupled to a particle, for example a microbead, that identifies the capture probe, for example using a fluorescent dye, under stringent conditions, preferably highly stringent conditions, very highly stringent conditions or extremely stringent conditions, by the hybridization tag. The hybridization of the extension product to the hybridization tag is detected using the detectable label and the identity and/or presence of the SNP is determined based on the identity of the particle coupled to the capture probe. That is, the particle identities which capture probe the hybridization tag is hybridized to, which in turn identifies the SNP, since the hybridization tag identifies the primer and therefore identifies the SNP.

In one embodiment, a plurality of different types of primers are used, each type specific for a different SNP. In another embodiment, groups of primers are used, each group comprising at least two primers specific for different alleles of a SNP. In yet another embodiment, the 3′ end of each primer is located immediately adjacent to the location of the SNP of interest. In still another embodiment, the primer is extended by a single base.

A further embodiment provides, a method for diagnosing a disease, condition, disorder or predisposition in a subject comprising, obtaining a biological sample containing a polynucleotide from the subject and analyzing the polynucleotide to detect the presence of absence of a single nucleotide polymorphism by any of the novel methods described herein, wherein the single nucleotide polymorphism is associated with a disease, condition, disorder or predisposition. In one embodiment, the biological sample is obtained from an animal or a plant, preferably a vertebrate animal or a vascular plant.

An additional aspect provides a method for selecting hybridization tags comprising identifying non-coding sequences of between about 10 to 100 nucleotides long, where the sequences lack hairpin structures and duplex-forming abilities. Non-coding sequences having a GC content of between about 40% to about 50% and a Tₘ that varies by no more than about 2° C. are further identified and these are selected for hybridization tags.

### BRIEF DESCRIPTION OF THE FIGURES

These and other features, aspects, and advantages of the present invention will become better understood with regard to the following description, appended claims and accompanying figures where:

FIG. 1 shows production of cDNA for expression profiling using the present inventive discovery.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, public databases, public database entries and other references cited in this application are herein incorporated by reference in their entirety as if each individual publication, patent, patent application, public database, public database entry or other reference were specifically and individually indicated to be incorporated by reference.

The present inventive discoveries provide novel methods for use in the analysis of nucleic acids. These methods are particularly useful for detecting the presence specific polynucleotides in complex samples and so are useful for expression analysis, such as gene expression analysis. In additional embodiments, the inventive discoveries provide methods for the detection of single nucleotide polymorphisms (SNPs). SNPs have a wide variety of uses including diagnosis of genetic diseases and predispositions in plants and animals, including humans, as well as uses to identify valuable phenotypes and in marker assisted selection. In addition to providing multiplex capabilities, the methods provided have the advantages of adaptability, easy of use, and cost effectiveness.

As used herein, “SNP” means single nucleotide polymorphism.

As used herein “polynucleotide” and “oligonucleotide” are used interchangeably and refer to a polymeric (2 or more monomers) form of nucleotides of any length, either ribonucleotides or deoxyribonucleotides. Whether or not specifically stated, polynucleotides and oligonucleotides are considered to have a 5′ end and a 3′ end. Although nucleotides are usually joined by phosphodiester linkages, the terms also include peptide nucleic acids such as polymeric nucleotides containing neutral amide backbone linkages composed of aminoethyl glycine units (Nielsen et al., Science, 254:1497, 1991). The terms refer only to the primary structure of the molecule. Thus, the terms include double- and single-stranded DNA and RNA as well DNA/RNA hybrids that may be single-stranded, but are more typically double-stranded. In addition, the terms also refer to triple-stranded regions comprising RNA or DNA or both RNA and DNA. The strands in such regions may be from the same molecule or from different molecules. The regions may include all or one or more of the molecules, but more typically involve only a region of some of the molecules. The terms also include known types of modifications, for example, labels, methylation, “caps”, substitution of one or more of the naturally occurring nucleotides with an analog, internucleotide modifications such as, for example, those with uncharged linkages (e.g. methyl phosphonates, phophotriesters, phosphoamidates, carbamates etc.), those containing pendant moieties, such as, for example, proteins (including for e.g., nucleases, toxins, antibodies, signal peptides, poly-L-lysine, etc.), those with intercalators (e.g., acridine, psoralen, etc,), those containg alkylators, those with modified linkages (e.g. alpha anomeric nucleic acids, etc.), as well as unmodified forms of the polynucleotide. Polynucleotides include both sense and antisense, or coding and template strands. The terms include naturally occurring and chemically synthesized molecules.

The term “detectable label” refers to a label which when attached, preferably covalently, provides a means of detection. There are a wide variety of labels available for this purpose including, without limitation, radioactive labels such as radionuclides, fluorophores or fluorochromes, peptides, enzymes, antigens, antibodies, vitamins or steroids. For example, radioactive nuclides such as ³²P or ³⁵S, or fluorescent dyes are conventionally used to label PCR primers. Chemiluminescent dyes can also be used for the purpose. The label can be attached directly to the molecule of interest or be attached through a linker. More specific examples of suitable labels include xanthine dyes, rhodamine dyes, naphthylamines, benzoxadiazoles, stilbenes, pyrenes, acridines, Cyanine 3, Cyanine 5, phycoerythrin conjugated streptavidin, Alexa 532, fluorescein, tetramethyl rhodamine, fluorescent nucleotides, digoxigenin, and biotin-deoxyuracil triphosphate. Likewise, in some embodiments, the nucleic acid can be labeled using intercalating dyes such as, for example, YOYO, TOTO, Picogreen, ethidium bromide, and the like. As used herein “sequence” means the linear order in which monomers occur in a polymer, for example, the order of amino acids in a polypeptide or the order of nucleotides in a polynucleotide.

As used herein, the term “subject” refers to any plant or animal.

As used herein, the term “animal” includes human beings.

As used herein, “primer” or “oligonucleotide primer” means an oligonucleotide, either naturally occurring, as in a purified restriction enzyme digest, or produced synthetically, that under the proper conditions, is capable of hybridizing to a template DNA or RNA molecule to initiate primer extension by polymerization, such as by a DNA-dependent DNA polymerase, a RNA-dependent RNA polymerase, or a RNA-dependent DNA polymerase, to produce a DNA or RNA molecule that is complementary to the template molecule. Primers are often between about 5 to about 50, typically between about 10 to about 30 and more typically between about 18 to about 25 nucleotides in length, and do not contain palindromic sequences or sequences resulting in the formation of primer dimers. Often primers are single stranded, however, double stranded primers may be used provided the primer is treated to separate the strands prior to being used for primer extension.

The term “hybridization” as used herein refers to a process in which a strand of nucleic acid joins with a complementary strand through base pairing. The conditions employed in the hybridization of two non-identical, but very similar, complementary nucleic acids varies with the degree of complementarity of the two strands and the length of the strands. Thus the term comtemplates partial as well as complete hybridization. Such techniques and conditions are well known to practitioners in this field.

As used herein, the term “primer pair” means two primers that bind to opposite strands of a nucleic acid molecule.

In one disclosed aspect, a method for determining expression of a target polynucleotide is provided. The polynucleotide can be DNA or RNA. Any of the various types of DNA and RNA can be used, for example, mRNA, cRNA, viral RNA, synthetic RNA, cDNA, genomic DNA, viral DNA, plasmid DNA, synthetic DNA, amplified DNA or any combination thereof. The polynucleotides can be obtained from any source containing nucleic acids. Sources typically include cells and tissues from prokaryotes and eukaryotes such as bacteria, yeast, fungi, plants and animals. Polynucleotides can also be obtained from viruses. By tissue is meant a plurality of cells that in their native state are organized to perform one or more specific functions. Non-limiting examples of tissues include muscle tissue, cardiac tissue, nervous tissue, leaf tissue, stem tissue, root tissue, etc. Cells from which target polynucleotides are obtained can be haploid, diploid, or polyploid.

In one embodiment, the target polynucleotide is cDNA produced by reverse transcription of mRNA, typically polyA mRNA. Methods for the production of cDNA from RNA are well known in the art and can be found in standard references such as Sambrook et al., Molecular Cloning, 3^{rd} ed., Cold Spring Harbor Laboratory Press, 2001; Ausubel et al., Short Protocols in Molecular Biology, 4^{th} ed., Wiley, 1999, Innis et al., PCR Protocols, Academic Press, 1990. For example, using standard methods well known in the art, total RNA is isolated from the cells or tissues of interest. The RNA can be used for first strand cDNA synthesis without further purification or polyA mRNA can be isolated using standard methologies known to those of ordinary skill in the art. Once obtained, the RNA, for example polyA mRNA, is combined with a first oligonucleotide primer under conditions that allow for hybridization of the primer to the RNA. In one embodiment, a portion of the primer is capable of hybridizing to the target polynucleotide. For example, when the target polynucleotide is a polyA mRNA, the first primer may comprise a portion containing a series of Ts, that is an oligo(dT) portion. In one embodiment, the first primer comprises the sequence 5′ attctagaggccgaggcggccgacatg-d(T)₃₀-vn-3′ (SEQ ID NO.: 1) where n is g, c, a, or t/u and v is g, c, or a. The conditions for hybridization are usually stringent conditions, often highly stringent conditions, very highly stringent conditions, or extremely stringent conditions.

As is well known in the art, stringency is related to the Tₘ of the hybrid formed. The Tₘ (melting temperature) of a nucleic acid hybrid is the temperature at which 50% of the bases are base-paired. For example, if one the partners in a hybrid is a short oligonucleotide of approximately 20 bases, 50% of the duplexes are typically strand separated at the Tₘ. In this case, the Tₘ reflects a time-independent equilibriun that depends on the concentration of oligonucleotide. In contrast, if both strands are longer, the Tₘ corresponds to a situation in which the strands are held together in structure possibly containing alternating duplex and denatured regions. In this case, the Tₘ reflects an intramolecular equilibrium that is independent of time and polynucleotide concentration.

As is also well known in the art, Tₘ is dependent on the composition of the polynucleotide (e.g. length, type of duplex, base composition, and extent of precise base pairing) and the composition of the solvent (e.g. salt concentration and the presence of denaturants such formamide). One equation for the calculation of Tₘ can be found in Sambrook et al. (Molecular Cloning, 2nd ed., Cold Spring Harbor Press, 1989) and is:

Tₘ=81.5° C.−16.6(log₁₀[Na⁺])+0.41(% G+C)−0.63(% formamide)−600/L)

Where L is the length of the hybrid in base pairs, the concentration of Na⁺ is in the range of 0.01M to 0.4M and the G+C content is in the range of 30% to 75%. Equations for hybrids involving RNA can be found in the same reference. Alternative equations can be found in Davis et al., Basic Methods in Molecular Biology, 2nd ed., Appleton and Lange, 1994, Sec 6-8.

Methods for hybridization and washing are well known in the art and can be found in standard references in molecular biology such as those cited herein. In general, hybridizations are usually carried out in solutions of high ionic strength (6×SSC or 6×SSPE) at a temperature 20-25° C. below the Tₘ. Specific examples of stringent hybridization conditions include 5×SSPE, 50% formamide at 42° C. or 5×SSPE at 68° C. Stringent wash conditions are often determined empirically in preliminary experiments, but usually involve a combination of salt and temperature that is approximately 12-25° C. below the Tₘ. One example of highly stringent wash conditions is 1×SSC at 60° C. An example of very highly stringency wash conditions is 0.1×SSPE, 0.1% SDS at 42° C. (Meinkoth and Wahl, Anal. Biochem., 138:267-284,1984). An example of extremely stringent wash conditions is 0.1×SSPE, 0.1% SDS at 50-65° C. In one preferred embodiment, high stringency washing is carried out under conditions of 1×SSC and 60° C. As is well recognized in the art, various combinations of factors can result in conditions of substantially equivalent stringency. Such equivalent conditions are within the scope of the present inventive discovery.

In one embodiment, following hybridization of the first oligonucleotide primer to the target RNA, a first strand cDNA is produced by providing dNTPs, a RNA-dependent DNA polymerase, such as a reverse transcriptase, and other necessary ingredients under conditions that allow for first strand cDNA synthesis by primer extension. Any reverse transcriptase capable of producing a cDNA molecule such as avian myeloblastosis viral (AMV) reverse transcriptase or Moloney murine leukemia virus (MMLV) reverse transcriptase can be used. Reverse transcriptases that lack or have reduced RNase H activity may be favorably employed in the present method. In a preferred embodiment, the reverse transcriptase used possesses terminal transferase activity. Terminal transferase activity refers to the ability of the polymerase to add nucleotides, primarily deoxycytidine, to the 3′ end of a polynucleotide independent of a template. This allows the production of a single-stranded extension that extends at least one nucleotide beyond the 5′ end of the template RNA In one embodiment, the reverse transcriptase used is PowerScript™ reverse transcriptase available from Clontech Laboratories, Inc (Palo Alto, Calif.).

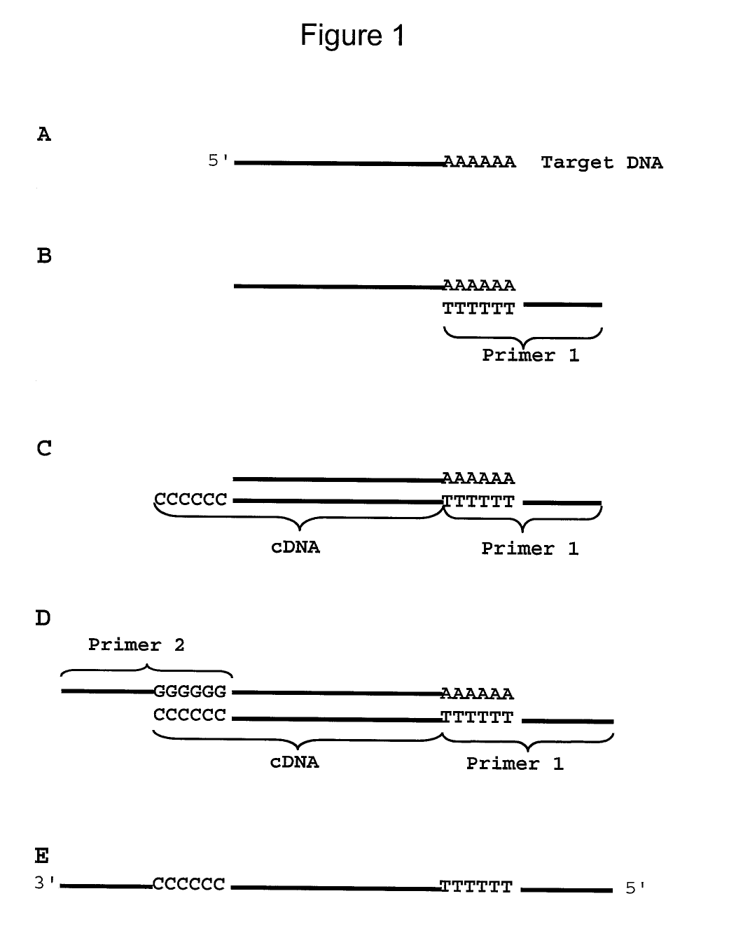

A second oligonucleotide primer is also provided. This primer is designed so that a portion of the primer is capable of hybridizing to the single-stranded extension of the first strand cDNA. The conditions for hybridization are usually stringent conditions, often highly stringent, very highly stringent, or extremely stringent conditions. In the situation where the single-stranded region is a dC or a poly d(C) region, the second primer comprises a polyG portion. In one embodiment, the second primer comprises the sequence 5′-aagcagtggtatcaacgcagactggccattacggccggg-3′ (SEQ ID NO.: 2). Synthesis of the first strand cDNA then continues using the second primer as a template. In one embodiment, this is accomplished by the reverse transcriptase present switching templates. The resulting cDNA molecule produced comprises the first primer, a portion complementary to the target polynucleotide, and a portion complementary to the second primer (FIG. 1E).

In one embodiment, the portion of the primers that is not complementary to either the target polynucleotide or the single-stranded extension, may contain at least one restriction endonuclease recognition site. The recognition site may be for the same restriction enzyme in both primers or each primer may have a different recognition site. The exact restriction enzyme recognition sites incorporated into the primer will vary with the particular use envisioned. Information on restriction enzyme recognition sites can be found in standard molecular biology texts such as those cited herein and in publicly available databases such as The Restriction Enzyme Database (rebase) which can be found at http://rebase.neb.com/rebase/. In one embodiment, a restriction endonuclease recognition site is chosen that does not appear in the target sequence. In another embodiment a recognition site is selected that appears rarely, if at all, in polynucleotides from the species from which the target polynucleotide was obtained. In one embodiment, the first primer comprises a SfiIB recognition site and the second primer comprises a Sfi IA recognition site.

The cDNA produced can then be amplified. Any method of amplification can be used including the polymease chain reaction (PCR) (U.S. Pat. Nos. 4,965,188; 4,800,159; 4,683,202; 4,683,195), ligase chain reaction (Wu and Wallace, Genomics, 4:560-569, 1989; Landegren et al., Science, 241:1077-1080, 1988), transcription amplification (Kwoh et al. Proc. Natl. Acad. Sci. USA, 86:1173-1177, 1989), self-sustained sequenced replication (Guatelli et al., Proc. Natl. Acad. Sci. USA, 87:1874-1878, 1990) and nucleic acid based sequence amplification (NASBA). In one embodiment, amplification is accomplished by PCR. Depending on the size of the target polynucleotide and the amount of starting material, PCR can be accomplished by long distance PCR (LD-PDR; U.S. Pat. Nos. 5,616,494 and 5,436,149; Barnes et al., Proc. Natl. Acad. Sci. USA, 91:2216-2220, 1994) or by conventional primer extension PCR (U.S. Pat. Nos. 4,965,188, 4,800,159, 4,683,202, 4,683,195). Primers used for PCR may be the same as used for first strand cDNA synthesis. Alternatively, truncated versions of the cDNA synthesis primers can be used or a combination of truncated and unaltered primers can be used. When primers are truncated, it is usually accompanied by removal that portion of the primer containing a single nucleotide. In one embodiment, the amplification primers used at SEQ ID NO.: 1 and the first 23 bases, counting from the 5′ end, of SEQ ID NO.: 2. Primers used may be present in equal or unequal amounts. The result of having unequal amounts of primers will result in increased amplification of one strand of a double-stranded polynucleotide relative to the other strand. As is well known in the art, the optimum conditions for PCR vary with such factors and the template sequences, the primers, and the polymerase used. Such optimization is considered routine in the art and can be accomplished by the skilled technician without undue experimentation.

During amplification, a detectable label or marker is incorporated into the amplification products. The label can be incorporated by using primers, one or both of which contain a label, labeled nucleoside triphosphates (NTPs), or a combination of labeled primers and NTPs. Those skilled in the art know which label should be used in conjunction with the particular experimental conditions employed. In certain embodiments, the labels are biotin-deoxyuracil triphosphate and phycoerythrin conjugated streptavidin. The label can be attached directly to the molecule of interest, be attached through a linker, or be located on a particle such as a microbead.

Optionally, the amplified and labeled cDNA may be fragmented by digestion with a suitable enzyme. The enzyme or enzymes used may be a random nuclease, such as a DNAse, or a non-random nuclease such as a restriction endonuclease. If a restriction endonuclease is used, it can have either a degenerate or non-degenerate recognition sequence. The terms “restriction endonuclease” and “restriction enzyme” are used interchangeably and in the broadest sense, and refer to an enzyme that recognizes a double-stranded DNA sequence-specifically and cuts it endonucleotically. It is noted that when a restriction endonuclease is referred to as a “four-base cutter”, “six-base cutter”, etc. reference is made to the number of nucleotide bases within the recognition sequence of such restriction endonuclease, not including degeneracy, if any. For example, a restriction endonuclease that has the degenerate recognition sequence CCNNGG, where “N” represents two or more of nucleotides A, G, C or T, would be referred to as a “four-base cutter”. Digestion with a “four-base cutter” restriction endonuclease will result in one cut approximately every 256 (4⁴) bases of the polynucleotide digested, while digestion with a “five-base cutter” restriction endonuclease will result in one cut approximately every 1024 (4⁵) bases, etc. Accordingly, one factor in choosing a restriction endonuclease will be the desired size and the number of the restriction endonuclease fragments for any particular application. When a random nuclease is used the size of the fragments will depend on well known factors such as the concentration of enzyme, the concentration of polynucleotide, time and temperature. The length of the digested cDNA is usually between about 50 to about 2000 bases, often between about 75 to about 1000 bases, typically between about 100 to about 1000 bases, more typically between about 150 to about 600 bases. Selection of appropriate restriction endonucleases and conditions for digestion with random nucleases can be made by one of ordinary skill in the art without undue experimentation. In one embodiment, digestion is accomplished using DNAse I.

Next the amplified, and optionally digested, labeled polynucleotides are hybridized to capture probes typically under stringent, more typically highly stringent, very highly stringent or extremely stringent conditions. Capture probes comprise polynucleotides of about 5 to about 100, often about 6 to about 75, more often about 8 to about 65, commonly about 10 to about 50, more commonly about 15 to about 40, typically about 16 to about 35, more typically about 18 to about 30 nucleotides in length. The capture probe contains a sequence complementary to a sequence on the target polynucleotide. The capture probe can be complementary to a sequence on the plus (coding, sense) strand, the minus (template, antisense) strand of the polynucleotide of interest or a combination of capture probes complementary to both strands of the target polynucleotide can be used. In one embodiment, the sequence on the target polynucleotide to which the capture probe hybridizes is unique to that target polynucleotide. In another embodiment, the capture probe hybridizes close to the 3′ end of the target polynucleotide, for example, within about 1000, about 800 or about 600 bases of the 3′ end. It will be apparent to those skill in the art, that multiple capture probes can be used for a single target polynucleotide.

Capture probes may be readily synthesized by well known techniques for the synthesis of polynucleotides such as those describe in U.S. Pat. No. 4,973,679; Gait, Oligonucletide Synthesis: A Practical Approach, IRL Press, 1984; Beaucage and Caruthers, Tetrahedron Letts, 22:1859-1862, 1981; Beaucage and Iyer, Tetrahedron, 48:2223-2311, 1992; Caruthers et al., Nucleic Acids Res. Symp. Ser., 7:215-223, 1980. Alternatively, capture probes may be custom ordered from numerous commercial sources. Capture probes are typically synthesized with a linker located on the 5′ end for attaching the probe to a solid substrate such as a particle. In one embodiment the 5′ amino uni-linker (Oligo Etc., Seattle, Wash.) is used.

The capture probes are couple to a solid substrate, for example a particle, that serves to identify the capture probe. In one embodiment, the substrate is a microbead or microsphere. The identity of the capture probe can be accomplished using microbeads of different sizes, shapes and/or colors (labels). The microbeads can range in size from about 0.1 micrometers to about 1000 micrometers, generally about 1 to about 100 micrometers, typically about 2 to about 50 micrometers, more typically about 3 to about 25 micrometers, usually about 6 to about 12 micrometers. The microbeads can be made of any suitable material including, but not limited to, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, polysulfone, or combinations thereof. Other polymer materials such as carbohydrate, e.g., carboxymethyl cellulose, hydroxyethyl cellulose, agar, gel, proteinaceous polymer, polypeptide, eukaryotic and prokaryotic cells, viruses, lipid, metal, resin, latex, rubber, silicone, e.g., polydimethyldiphenyl siloxane, glass, ceramic, charcoal, kaolinite, bentonite, and the like can also be used. In one embodiment, commercially available Luminex microbeads (Luminex Corp., Austin, Tex.) are used.

Luminex microbeads are extensively discussed in U.S. Pat. No. 6,268,222 and PCT publications WO 99/37814 and WO 01/13120. Briefly, the microbeads are microparticles that incorporate polymeric nanoparticles stained with one or more fluorescent dyes. All of the nanoparticles in a given population are dyed with the same concentration of a dye, and by incorporating a known quantity of these nanoparticles into the microsphere, along with known quantities of other nanoparticles stained with different dyes, a multifluorescent microsphere results. By varying the quantity and ratio of different populations of nanoparticles it is possible to establish and distinguish a large number of discrete populations of microspheres with unique emission spectra. The fluorescent dyes used are of the general class known as cyanine dyes, with emission wavelengths between 550 nm and 900 nm. These dyes may contain methine groups; the number of methine groups influences the spectral properties of the dye. The monomethine dyes that are pyridines typically have a blue to blue-green fluorescence emission, while quinolines have a green to yellow-green fluorescence emission. The trimethine dye analogs are substantially shifted toward red wavelengths, and the pentamethine dyes are shifted even further, often exhibiting infrared fluorescence emission. However, any dye compatible with the composition of the beads can be used.

When a number of different microbeads are used in practicing the methods described herein, it is preferable, but not required, that the dyes have the same or overlapping excitation spectra, but possess distinguishable emission spectra. Multiple classes or populations of particles can be produced from just two dyes. The ratio of nanoparticle populations with red/orange dyes is altered by an adequate increment in proportion so that the obtained ratio does not optically overlap with the former ratio. In this way a large number of differently fluorescing microbead classes are produced.

Capture probes are then coupled to the microbeads. The exact method of coupling will vary with the composition of the microbread and the type of linker present, if any. In one embodiment, capture probes are coupled to microbeads by the well known carbodiimide coupling procedure. Multiple capture probes are coupled to a single microbead. Microbeads of the same class or group, that is having the same label or fluorescent signature, will have capture probes specific for the same target polynucleotide attached to them. The sequence of capture probes attached to a single microbead or class of microbeads may be the same of different. For example, capture probes complementary to the coding strand, the template strand or a combination thereof may be attached to a single microbead or class of microbeads. Likewise a single microbead or class of microbeads may comprise capture probes complementary to different regions of the same target polynucleotide.

Any detection system can be used to detect the difference in spectral characteristics between the two dyes, including a solid state detector, photomultiplier tube, photographic film, or eye, any of which may be used in conjunction with additional instrumentation such as a spectrometer, luminometer microscope, plate reader, fluorescent scanner, flow cytometer, or any combination thereof, to complete the detection system.

When differentiation between the two dyes is accomplished by visual inspection, the two dyes preferably have emission wavelengths of perceptibly different colors to enhance visual discrimination. When it is desirable to differentiate between the two dyes using instrumental methods, a variety of filters and diffraction gratings allow the respective emission maxima to be independently detected.

In one embodiment microbeads are identified using a flow cytometer, for example a fluorescence-activated cell sorter, wherein the different classes of beads in a mixture can be physically separated from each other based on the fluorochrome identity, size and/or shape of each class of bead, and the presence of the target polynucleotide qualitatively or quantitatively determined based on the presence of the detectable label for each sorted pool containing beads of a particular class. Any flow cytometer capable of detecting both the particles and the label contained in the polynucleotide hybridized to the capture probe can be used. Flow cytometers with multiple excitation lasers and detectors are preferred. In one embodiment the Luminex 100 flow cytometer is used. As is well known in the art, the exact setting necessary for optimum detection will vary with factors such as the flow cytometer used, the polynucleotide label used, and the particles used. Optimization of settings and conditions for the use of a flow cytometer for practicing the methods disclosed herein can be accomplished by the skilled technician without undue experimentation. General guidance on the use of flow cytometers can be found in texts such as Shapiro, Practical Flow Cytometry, 3^{rd} ed., Wiley-Liss, 1995 and Jaroszeski et al., Flow Cytometry Protocols, Humana Press, 1998. An example of the use of fluorescent microbeads and flow cytometery can be found in Smith et al., Clin. Chem., 44:2054-2056, 1998. The use of flow cytometry is especially useful in the situation where greater than one class of particles and a plurality of capture probes are used to simultaneously to determine the presence of multiple target polynucleotides (multiplex analysis).

Determination of the presence and/or amount of target polynucleotide present is accomplished using a combination of the signals from the particles and the labeled target polynucleotide. The particle is used to identify a particular capture probe specific for a given target polynucleotide, for example by the fluorescent signature of a microbead. The identified capture probe is then analyzed to determine the presence of the label contained in the target polynucleotide. If the label is present on the capture probe then the target polynucleotide is present in the sample. By quantification of the amount of label present, the amount of target polynucleotide in the sample can be calculated.

The ability to simultaneously determine the presence and/or amount of a target polynucleotide make the present methods especially suitable for expression profiling. Expression profiling involves the determination of changes in the expression of polynucleotides, e.g. genes, under different conditions and physiological states. Thus, the methods described herein are useful for diagnosing a disease, condition, disorder or predisposition associated with a change in expression patterns. As used herein, the term “predisposition” refers to the likelihood that an individual subject will develop a particular disease, condition or disorder. For example, a subject with an increased predisposition will be more likely than average to develop a disease, condition or disorder, while a subject with a decreased predisposition will be less likely than average to develop a disease, condition or disorder. The disease, condition, disorder, or predisposition may be genetic or may be due to a microorganism. In this aspect, information on the expression of one or more target polynucleotides is obtained from an test subject using the methods described herein and compared to the expression pattern for a subject known to have the disease, condition, disorder or predisposition of interest. In one embodiment, data representing the expression pattern of a subject with a known disease, condition, disorder or predisposition is stored on a computer readable medium so that the expression pattern from the test subject can be compared to the stored expression pattern.

Likewise the methods disclosed herein can be used to determine the developmental or physiological state of a cell or tissue. In this aspect, polynucleotide expression from a test cell or tissue is compared to the expression pattern from a cell of known physiological or developmental state. By comparing the two expression patterns, it is possible to determine the developmental or physiological state of the test cell or tissue. In one embodiment, data representing the expression pattern of a cell or tissue of a known developmental or physiological state is stored on a computer readable medium so that the expression pattern from the test cell or tissue type can be compared to the stored expression pattern.

Another aspect of the present inventive discovery provides an inexpensive, fast, flexible method for SNP analysis that is suitable for high throughput applications. Using the present methods, identification of SNP alleles is possible by either a PCR strategy such as allele specific PCR (ASP) or by a simple primer extension methodology such as short primer extension (SPE). In both cases, the amplification or extension is conducted in the presence of a detectable label.

When utilizing PCR-based methods such as ASP, at least one pair of primers is used. Each primer pair contains a forward primer and a reverse primer. The 3′ end of the forward primer of the pair is specific for an allele of the SNP of interest, for example, the 3′ end of the primer contains a nucleotide complementary to allelic bases of the SNP of interest. The forward primer may also contain a hybridization tag that identifies the primer and that is not complementary to the polynucelotide containing the SNP of interest. The hybridization tag is typically located on the 5′ end of the primer. The reverse primer can be specific for the polynucleotide containing the SNP of interest, or it can be a universal reverse primer. The reverse primer can also contain a hybridization tag. In one embodiment, primer pairs specific for each possible SNP allele are used. In another embodment, multiple primer pairs specific for multiple SNPs are used i.e. multiplex analysis. When multiplex analysis is being used, a single primer pair for each SNP can be used or primer pairs corresponding to the possible alleles for the various SNPs of interest can be used. The use of primer pairs for each SNP allele aids in the determination of individuals heterozygous for the SNP or SNPs or interest.

The primer pairs are then combined with a sample containing one or more single-stranded polynucleotides. The polynucleotides may be RNA, DNA or a combination thereof. Particular examples include, but are not limited to, mRNA, cRNA, viral RNA, synthetic RNA, cDNA, genomic DNA, viral DNA, plasmid DNA, synthetic DNA, amplified DNA or any combination thereof. If the polynucleotides present in the sample are double-stranded, they can be made single-stranded using well known methods in the art such as chemical or heat treatment. The primers are allowed to hybridize to the single-stranded polynucleotides under stringent typically highly stringent conditions, very highly stringent conditions or extremely stringent conditions. Typically stringent hybridization conditions are adjusted so that a single mismatch on the 3′ end of the forward primer will prevent or significantly reduce hybridization. By “significantly reduce”, is meant that hybridization is reduced at least 50%, typically at least 75%, more typically at least 85%, commonly at least 90%, more commonly at least 95% and preferably at least 99% when compared that observed with a perfectly complementary sequence. As is well known in the art, specific conditions for hybridization are typically determined empirically and can be accomplished by one of ordinary skill in the art without undue experimentation using the guidance provided herein as well as standard texts on molecular biology, such as those cited herein.

Once hybridized, the primers are extended to produce an extension product. In one preferred embodiment, primer extension is repeated several times as in PCR to produce large amounts of extension product. Methods for primer extension, especially PCR are well known in the art and have been discussed herein. In general the method involves supplying a polymerase, often a heat stable polymerase, dNTPs and necessary cofactors followed by a series of hybridization, extension and denaturation steps. The hybridization products produced contain the hybridization tag and a detectable label. The label can be incorporated into the extension products by using a labeled primer, labeled dNTPs or a combination thereof. Any detectable label suitable for use with polynucleotides can be used, including those described previously.

Once produced, the extension products are hybridized under stringent, highly stringent, very highly stringent or extremely stringent conditions to a capture probe that is complementary to the hybridization tag or the complement thereof. The capture probe is in turn coupled to a solid substrate, for example a microbead. The particle contains a dye or other substance to provide a detectable signal or signature specific for the class of particle containing a particular capture probe. In the case where the particle contains a fluorescent dye, the particle has a unique fluorescent signature. The use of labeled particles, and in particular microbeads containing fluorescent dyes has been described previously. In one embodiment, fluorescent microbeads commercially available for the Luminex Corp. are used.

The combination of the label incorporated into the extension products and the particles are then used to identify the SNP present. For example, when fluorescent microbeads are used, the microbeads are identified and optionally separated on the basis of their fluorescent signature. The signature identifies the capture probe attached to the bead, which in turn identifies the hybridization tag that identifies the SNP of interest. The bead is also examined for the presence of the label incorporated into the extension products. If the label is present, then the extension was present indicating successful hybridization of the forward primer and thus the presence of a particular SNP allele. When only one primer per SNP is used, then the absence of the label suggests that the alternative SNP allele is present. In one embodiment, this analysis is carried out using a flow cytometer. The use of flow cytometers to identify labeled beads and associated polynucleotides has been discussed previously.

In an alternative embodiment, short primer extension (SPE) is used instead of allele specific PCR. In this embodiment, at least one primer whose 3′ end is specific to the SNP of interest is provided. This primer also contains a hybridization tag. In this embodiment, a primer is considered specific for a SNP when it contains on its 3′ end a base complementary to one of the allelic bases of the SNP of interest. Alternatively, a primer is considered specific for a SNP when it specifically hybridizes such that its 3′ end is immediately adjacent to the location of the SNP of interest, such that the addition of the next base to the 3′ end of the primer will be directed by the base present at the location of the SNP. The primer is combined with a single-stranded polynucleotides under stringent conditions that allow specific hybridization of the primer or primers to the single stranded polynucleotides. If the sample used contains double-stranded polynucleotides then they are made single-stranded prior to primer hybridization. If primers are used which extend to the site of the SNP, the hybridization conditions are adjusted so that a single mismatch on the 3′ end of the primer will prevent or significantly reduce hybridization as discussed previously. If the primer used hybridizes immediately adjacent to the SNP location, then hybridization conditions are adjusted to minimize non-specific hybridization.

The primer or primers are then extended using a polymerase and suitable nucleoside triphosphates (NTPs). In one embodiment, primer extension is limited by the inclusion of one or more chain terminating nucleoside triphosphates, such as dideoxynucleotide triphosphates (ddNTPs), in the reaction mix. In embodiments where the primer or primers hybridize immediately adjacent to the SNP location, a single type of NTP can be added to that primer extension occurs only if the complementary allele is present (see U.S. Pat. No. 5,846,710). Alternatively, only chain-terminating NTPs, such as ddNTPs, can be used so that only a single base is added to the 3′ end of the primer. When this alternative is used, preferably each ddNTP contains a different detectable label (see U.S. Pat. No. 5,888,819). The extension product produced comprises a hybridization tag and a detectable label. The label can be incorporated into the extension product using a labled primer, labeled NTPs or a combination thereof. Any of the labels previously discussed can be used.

The labeled extension product is then hybridized under stringent, highly stringent conditions, very highly stringent conditions or extremely stringent conditions to a capture probe which is complementary to the hybridization tag. The capture probe is coupled to a particle such as those discussed previously that identifies the capture probe. The identity of the SNP allele present is then made by detecting the hybridization of the extension product to its specific capture probe and identifying the capture probe on the basis of the particle as has been discussed previously. Any of the previously discussed methods of detection can be used including flow cytometry.

The methods described herein for the detection of SNPs have widespread application. For example, the methods can be used to screen individuals for a genetic predisposition to a disease, condition or disorder of interest. In this aspect, a biological sample containing polynucleotides is obtained from a test subject. The polynucleotides contained in the sample are then analyzed using the instant methods to detect the presence of a SNP or SNPs associated with the disease, condition or disorder of interest.

Detection of SNPs using the methods disclosed herein can also be used in marker assisted selection. SNPs have been associated with various traits in plants and animals. Especially useful are SNPs located in quantitative trait loci (QTLs), By practicing selection on the presence of absence of a particular SNP, genetic progress can be achieved more rapidly than by traditional selection methods based on measurement of phenotype.

### EXAMPLES

The following examples are intended to provide illustrations of the application of the present invention. The following examples are not intended to completely define or otherwise limit the scope of the invention.

### Example 1

### Expression Analysis

1.1 Total RNA Extraction and PCR Products Labeling

Frozen tissue samples were homogenized in 96-well plates using an automated tissue disruption machine. Total RNA was extracted from the tissue homogenates using the Bioline 96 well RNA kit (Bioline, Boston Mass.) following the manufacturer's protocols and quantified by absorbance at 260 nm. Total RNA (1 μg) of each sample was converted to cDNA via the SMART kit (Clontech, Palo Alto, Calif.). The cDNA was then PCR amplified in 27 cycles by the same kit following the manufacturer's protocol and labeled with biotin-dUTP. The PCR DNA was next fragmented by 1U Dnase I at room temperature for 7 min. The reaction was stopped by heating at 95° C. for 10 min.

1.2 Capture Probe and Its Coupling to Microspheres

A unique sequence of 25 bases within a region of 600 bases from the 3′-end of a target gene was chosen as a capture probe. Multiple capture probes could be selected from a same polynucleotide or gene close to the 3′-end at different positions. The melting temperature (Tₘ) of the chosen capture probe usually ranged from 50° C. to 65° C. and the secondary structure was preferred minimal (Vector NTI, North Bethesda, Md.). All capture probe oligonucleotides were synthesized with 5′-amino uni-linker (Oligos Etc., Seattle, Wash.) and then covalently linked to carboxylated fluorochrome microspheres (Luminex Corp., Austin, Tex.). Specifically, 5×10⁶ of carboxylated microspheres were centrifuged in a microcentrifuge for 1 min at maximum speed and the supernatant was carefully removed by a pipette without disturbing the microspheres. The microspheres were resuspended in 50 μL of buffer containing 0.1 M MES (2-(N-morpholino)ethanesulfonic acid)(Sigma, St. Louis, Mo.), pH 4.5. The amino-substituted capture probe was dissolved in ddH₂O at a concentration of 1 mM and 1 μL of the solution (containing 1 nmol of capture probe oligonucleotides) was added to the microspheres for the coupling reaction. The coupling reaction was initiated by adding 2.5 μL of freshly made 10 mg/mL of 1-ethyl-3-(3-dimethylaminopropyl) carbodiimide hydrochloride (EDC) (Aldrich, Milwaukee, Wis.) that was dissolved in ddH₂O. The mixture of microspheres, capture probe, and EDC was briefly vortexed and incubated at room temperature for 30 min in the dark. After the 30-min incubation, a second 2.5-μL of a newly prepared EDC solution (10 mg/mL) was added to the reaction and incubated for an additional 30 min. This step was repeated for a total of three times (three EDC additions). During the incubations, the reaction was occasionally mixed by finger flicking the tube to keep the microspheres in suspension. After the coupling reaction, 1 mL of 0.02% Tween 20 (BioRad, Hercules, Calif.) was added to the microspheres. The solution was mixed well and centrifuged in a microcentrifuge for 1 min at maximum speed. The supernatant containing free capture probe oligonucleotides and excess EDC was carefully removed. The microspheres were washed again in 1 mL of 0.1% SDS (Ambion, Austin, Tex.) to ensure the removal of free capture-probe and EDC. In the end, the capture probe conjugated microspheres were resuspended in 100 μL of a buffer containing 0.1 M of MES, pH 4.5. The coupled microspheres were stored at 4° C. in a dark box and stayed stable for at least six months. The microspheres were diluted in TE buffer (10 mM Tris, 1 mM EDTA) and numerated in a cell counter slide under 100× magnification. For a single hybridization assay, about 7500 of coupled microspheres of each set were used. The coupling efficiencies and hybridization specificity were evaluated by hybridizing the coupled microspheres to their corresponding biotinylated complementary oligonucleotides.

1.3 Hybridization of Targets to Capture Probes Coupled Microspheres

The 1× hybridization buffer contained 3M tetramethylammonium-chloride (TMAC, Sigma, St. Louis, Mo.), 0.1% of SDS, 50 mM of Tris-HCl pH 8.0 and 4 mM of EDTA pH 8.0. The stock hybridization solution was prepared as 1.5× and stored at 50° C. to prevent precipitation. In the first step, target samples containing PCR DNA fragments in 20 μL were denatured by heating at 95° C. for 10 min. Capture probe conjugated microspheres (about 7500 beads per color) were mixed in 40 μL of 1.5× hybridization buffer and subsequently added to the denatured target samples. The hybridization mixture was quickly vortexed and incubated at 48° C. for 1 h in an Eppendorf microtube incubator (Eppendorf Scientific, Inc., Westbury, N.Y.) with a shaking speed of 300 rpm. After incubation, the hybridization mixture was centrifuged for 1 min at 14,000 g in a microcentrifuge. Supernatant was carefully removed with a pipette without disturbing the microspheres. The microspheres were washed by adding 50 μL of 1× hybridization solution, mixed by finger flicking and incubated at 48° C. for 5 min without shaking and spun for 1 min at maximum speed and remove supernatant. After the microspheres were washed for a total of three times, 50 μL of 133 TMAC and 0.5 μL of 1 mg/ml of streptavidin conjugated R-phycoerythrin (Molecular Probes, Inc., Eugene, Oreg.) was added to the microspheres. The solution was briefly vortexed and incubated in the dark for 10 min at room temperature. The microspheres (35 μL) were analyzed on the Luminex¹⁰⁰ system and at least 200 events of each set of microspheres were counted.

Representative results obtained using the method of Example 1 are shown in Table 1. In this example, differences in expression of 11 different genes in response to 3 chemical treatments were obtained. Expression levels are given in units of mean fluorescent intensity (MFI).

TABLE 1

| | Expression Level (MFI) | | |
|---|---|---|---|
| Gene | Treatment 1 | Treatment 2 | Treatment 3 |
| sbe2-1 | 69 | 304 | 147 |
| SUGTL1 | 213 | 1036 | 445 |
| pas | 183 | 721 | 362 |
| CAC2 | 80 | 200 | 123 |
| ATP8a | 161 | 425 | 353 |
| CAD1 | 142 | 394 | 214 |
| CPN10 | 123 | 185 | 165 |
| HSP70 | 214 | 2710 | 716 |
| HSC17.6 | 54.5 | 259 | 135 |
| AK22 | 134 | 467 | 236 |
| ATPK15D | 123 | 314 | 202 |

### Example 2

### SNP Analysis

2.1 Design of Universal Hybridization Tags (UHTs)

A series of DNA hybridization tags were derived for conjugation to Luminex microspheres. The DNA hybridization tags were named “Universal Hybridization Tags” (UHTs) because the DNA tags (in the form of oligonucleotides) and microspheres could be used for any SNP marker assay depending on whether the UHT was incorporated into the design of the SNPs primer sequences. To derive a source of random DNA sequence from which to generate the UHT sequences, a non-coding intron DNA sequence from an organism that would not be used subsequently was chosen. Another option for generating a random series of 18mers that would have good hybridization characteristics associated with them requires software capable of analyzing a large amount of DNA sequence. The intronic DNA sequence was chosen because it would not have the high degree of selective pressure that is found in the coding DNA, increasing the random nature of the DNA sequence. This was important to ensure there would be minimal non-specific interaction interfering with the assay's integrity.

The intronic DNA sequence chosen was from the large 50 kb intron 3 of the Drosophila ubx locus found in Genbank accession #U31961. The 3^{rd} intron of the Ubx gene containing approximately 50 kb of DNA sequence was imported into the OLIGO 5.0 software. The search algorithm was then customized to search for DNA sequences with Tₘₛ of about 60° C. and optimally 18 bases in length that lacked hairpin structures and duplex forming abilities. Additionally, the melting temperature (Tm) was set so that each UHT sequence varied by no more than 2° C. and contained a “GC” content of about 40 to 50%. With these settings, it was hypothesized that the resulting characteristic of all of the UHT sequences would be very specific hybridization in the same temperature range with a lack of background artifacts. The raw output derived from the DNA sequence search was analyzed and trimmed to contain a list of unique UHT DNA sequences (Table 2) SEQ ID NOS:3-46).

Oligonucleotides used had the exact UHT DNA sequence and possessed a Unilinker amino-linker modification on the 5′ end to allow conjugation to carboxylated microspheres. Also, biotin-labeled oligonucleotides having the complementary sequence were obtained to measure hybridization specificity. A Luminex hybridization experiment was designed to test each UHT sequence with its specific biotinylated oligonucleotide and a mixture of non-specific oligonucleotides. The signals were measured from each condition. The criterion for approval was a specific signal of 3000 fluorescence units or higher and a S/N ratio of above 35 (see Table 2).

TABLE 2

| | | | | | |
|---|---|---|---|---|---|
| | | SEQ | | | |
| | | ID | Specific | S/N | Ap- |
| UHT # | Sequence | NO | Signal | Ratio | proved |

| | | | | | |
|---|---|---|---|---|---|
| 1 | aaaacatccttccaccga | 3 | 3597.5 | 36.9 | Y |
| 2 | gtccttctgtccgctcaa | 4 | 3102.5 | 43.7 | Y |
| 3 | ggcggaatgagatacgat | 5 | 6576.5 | 144.5 | Y |
| 4 | tcgcactttttcgcataa | 6 | 9080.5 | 201.8 | Y |
| 5 | accgactggaaccgaata | 7 | 989.0 | 8.7 | N |
| 6 | gcaaaacaatggcgagta | 8 | 7574.5 | 14.0 | N |
| 7 | tggtctggtctggtctgg | 9 | 8351.5 | 245.6 | Y |
| 8 | gaaaagcaaaccaaaccc | 10 | 423.5 | 3.6 | N |
| 9 | accctcctctccacgatt | 11 | 829.0 | 20.7 | N |
| 10 | aaggggatgggaaagtct | 12 | 3604.0 | 92.4 | Y |
| 11 | ttcctcttcctcttgcca | 13 | 3362.0 | 86.2 | Y |
| 12 | tgcggctggacttactct | 14 | 6515.0 | 160.9 | Y |
| 13 | agccacagcccagtttag | 15 | 5988.5 | 88.7 | Y |
| 14 | attgaagcccgaacagac | 16 | 3029.5 | 59.4 | Y |
| 15 | ggctgcgttcaatcatct | 17 | 7402.0 | 185.1 | Y |
| 16 | ataccaaaaagcgagcct | 18 | 10813.0 | 277.3 | Y |
| 17 | tactaacgcccctggtct | 19 | 6668.5 | 43.3 | Y |
| 18 | gcccctgactcttgctaa | 20 | 9061.5 | 38.9 | Y |
| 19 | cttggtcggtcctttttg | 21 | 5302.0 | 88.4 | Y |
| 20 | agcggtgagtggagaaaa | 22 | 8781.0 | 117.9 | Y |
| 21 | tcgtcgtttgggtctctt | 23 | 8270.5 | 22.1 | N |
| 22 | gtggtggggttgtgagaa | 24 | 6234.5 | 55.9 | Y |
| 23 | aaacgaaacggaaccact | 25 | 11225.5 | 270.5 | Y |
| 24 | ccacgcacaaaaagaatc | 26 | 7562.5 | 162.7 | Y |
| 25 | tttggtttgggcttgtct | 27 | 7838.0 | 4.9 | N |
| 26 | cgatgttgcccctactgt | 28 | 4082.5 | 88.8 | Y |
| 27 | ttcgctgtggctctgtta | 29 | 7530.5 | 140.8 | Y |
| 28 | tcagttttccgcatttca | 30 | 4511.5 | 91.1 | Y |
| 29 | tattcaaaacgggaggct | 31 | 8976.0 | 138.1 | Y |
| 30 | ttgggtggcagataggtc | 32 | 3903.5 | 73.0 | Y |
| 31 | ttgtttttgggggtaggt | 33 | 3675.5 | 14.4 | N |
| 32 | agggtggaaaatgcgata | 34 | 7963.0 | 9.6 | N |
| 33 | agagtggcgagtgtaggg | 35 | 6553.0 | 20.9 | N |
| 34 | ataaggacccagccacaa | 36 | 8267.0 | 97.8 | Y |
| 35 | atcggctggcaataagtc | 37 | 921.0 | 9.4 | N |
| 36 | aggcaagtggagcagtqt | 38 | 6966.0 | 94.1 | Y |
| 37 | agagaaacggcacccata | 39 | 7965.5 | 17.9 | N |
| 38 | tccttcttggtctcgctt | 40 | 7599.5 | 47.2 | Y |
| 39 | aggaaaaagccatcgtca | 41 | 9323.5 | 46.6 | Y |
| 40 | acggagaatggcgagata | 42 | 8294.5 | 41.3 | Y |
| 41 | tgaccttgctgacccttt | 43 | 5332.5 | 92.7 | Y |
| 42 | tgttgtgcgtgttggaag | 44 | 3329.5 | 21.6 | N |
| 43 | gtttttgtgcctttcggt | 45 | 7797.5 | 134.4 | Y |
| 44 | gagtttctggagcggttg | 46 | 6520.0 | 26.4 | N |

2.2 Vegetable Marker “A” (NVMA, VegA) Primer Organization

The Vegetable Marker “A” is a combination of two proximal SNPs that are in complete linkage disequilibrium. Although it is a complicated concept, basically Linkage disequilibrium has to do with comparing two genetic markers that are physically linked by a DNA strand. For example, an unmutated DNA strand will contain the first SNP marker having two alleles, SNP1, (or two alternative bases at the same position) on a double-stranded DNA molecule. As time proceeds, the second SNP will occur and since it is a single event on the same single strand of DNA, the new SNP2 allele will be associated with only one of the alleles of SNP1. As time proceeds and many recombination events occur between the two SNPs, allele 1 of SNP2 can be associated with either allele of SNP1. At this time, SNP2 is in complete linkage disequilibrium with SNP1. As time again proceeds and many recombination events occur in between the two SNPs, allele 1 of SNP2 can be associated with either allele of SNP1 through haplotype analysis, and allele 2 of SNP2 again can be found with both alleles of SNP 1. When these association numbers equal 50%, then the two SNPs are in complete linkage equilibrium. Linkage generally proceeds toward an equilibrium. However, varying degrees of each condition can be found when comparing two genetic markers.

The gel based assay for VegA uses two forward primers at the NVMA-3 and NVMA-4 positions and the universal reverse PCR primer, NVMA-2.

| | | |
|---|---|---|
| NVMA-1 | 5′-ggattgcccaatacttaacact-3′ | SEQ ID NO:47 |
| NVMA-2 | 5′-acaagcctgctttggtgtgt-3′ | SEQ ID NO:48 |
| NVMA-3 | 5′-ctggagcgtggacaatatg-3′ | SEQ ID NO:49 |
| NVMA-4 | 5′-caagaaccctttcctcttcc-3′ | SEQ ID NO:50 |

When the PCR samples are electrophoresed on a gel, they give certain DNA band patterns that allow the genotype to be assigned. A similar assay set-up is used with the Luminex ASP procedure that allows direct comparison of the gel genotypes to the data output from the Luminex 100. When performing the Luminex SPE assay, the genomic DNAs were first amplified with NVMA-1 and NVMA-2 and followed up with primer extension using both the NVMA-3 and NVMA-4 together in the same reaction.

To perform the Short Primer Extension (SPE) procedure, an initial amplification step with standard PCR primers was performed, followed by primer extension with allele-specific extension primers containing UHT tags. The forward PCR primer was NVMA-1 and the reverse PCR primer was NVMA-2. The NVMA-3 primer is the extension primer specific for allele 1 while the NVMA-4 primer is the extension primer specific for allele 2. The first 18 bases of the NVMA-3 primer was tagged with the UHT #1 sequence and similarly, the 5′ end of the NVMA-4 primer with UHT #2 sequence.

The Allele-Specific PCR (ASP) procedure is a more simplified approach and only requires a PCR amplification using two allele-specific forward primers containing a C12 linker between the UHT and the marker related sequences, and one biotin-labeled universal reverse primer. The NVMA specific forward primers used were NVMA-3 and NVMA-4, representing the allele 1 and allele 2 genotypes, respectively. The universal reverse primer was NVMA-2. When performing the ASP assay approach, the samples used in this procedure were electrophoresed on an agarose gel, directly genotyped, and compared to the results obtained from the fluorescent microsphere procedure.

2.3 Testing of the UHTs

Before using the SNP assay, the UHT sequences were tested for their performance as useful DNA molecular tags using a series of synthetic oligonucleotides in conjunction with the Luminex system. For each UHT sequence, a forward strand oligonucleotide was obtained with a 5′ Unilinker label (Oligo Etc., Wilsonville, Oreg.) and a reverse strand was synthesized with a 5′ biotin label (Life Technologies. Rockville, Md.). Many amino linkers are available for conjugating oligonucleotides to the microspheres, but the Unilinker produced the best conjugation efficiency and consistency.

The Unilinker-labeled UHT oligonucleotides were conjugated to 1.25×10⁶ Development Microspheres. The Development Microspheres are generally used for initial testing of certain assay systems, because they tend to be less expensive then the multiplexing microspheres. They are, however, only one color so that assays can not be multiplexed using them. The UHT oligonucleotides were conjugated to the Development microspheres using a carbodiimide coupling procedure as follows:

2.3.1 Oligonucleotide/Microsphere Carbodiimide Conjugations

The Luminex microspheres were vortexed and sonicated for 10 seconds followed by centrifugation at 8000 g for 1.0 min. The supernatant was removed from the microsphere pellet and the microspheres were resuspended with MES buffer (0.1M (2[N-Morpholino]ethanesulfonic acid, 150 mM NaCl, pH to 4.5 with 5.0N KOH) at 2.5×10⁴ or 1.0×10⁵ microspheres/μl. For each coupling reaction, 50 μl of the microsphere/MES suspension was placed into a microcentrifuge tube along with 1.0 μl of a 1.0 mM solution (in H₂O) of the Unilinker labeled oligonucleotide (Oligo Etc.). 2.5 μl of freshly made 10 mg/ml EDC (1-ethyl-3-(3-dimethylaminopropyl)-carbodiimide hydrochloride) (Pierce Chemical Co., Rockford, Ill.) was added and incubated at room temperature for 30 min. Another 2.5 μl of a new batch of 10 mg/ml EDC solution was added and incubation was continued for another 30 min. One ml of MES buffer/0.02% Tween 20 was added and the microspheres were centrifuged at 8000 g for 1.0 min. The supernatant was removed and the pellet containing the microspheres was washed once more with 1.0 ml of the MES/Tween solution. This was followed by two 1.0 ml washes of the microspheres with MES buffer/0.1%SDS solution. Lastly, the microspheres were resuspended in 100 μl of MES buffer and enumerated.

The Development Microspheres are typically not used for multiplexing but are useful for general testing purposes such as this uniplex format. To test the conjugation efficiency along with the potential usefulness of each UHT DNA sequence, a Luminex hybridization assay was performed as follows:

2.3.2 Luminex Hybridization Assay

The synthetic oligonucleotide and/or genotyping samples of interest were made up to a volume of 20 μl with TE buffer (10 mM Tris-Cl, 0.1 mM EDTA, pH, 7.5). They were denatured for 10 min at 95° C. on a dry heat block apparatus. To the samples, 35 μl of a 1.5×TMAC (tetramethylammonium chloride) buffer (4.5 M TMAC, 0.15% SDS, 75 mM Tris-Cl, 6.0 mM EDTA, pH, 8.0) was added containing 5-10,000 microspheres/assay. The samples then were immediately placed at a 55° C. hybridization temperature in another heatblock for 10 min. A 1:400 dilution of a streptavidin/phycoerythrin conjugate (SA/PE, Molecular Probes; Eugene, Oreg.) was made with 1.0×TMAC (3.0M TMAC, 0.1% SDS, 50.0 mM Tris-Cl, 4.0 mM EDTA, pH, 8.0) and 50 μl was added to each sample. (If the samples had direct fluorescent labeling and biotin was not used in the assay, 50 μl of 1.0×TMAC alone without SA/PE, was added). The samples were additionally incubated at 55° C. for 5 min more and then analyzed in the Luminex 100 instrument.

For each UHT test, there was a positive control sample consisting of an exact oligonucleotide complement of the UHT DNA sequence, and a negative control consisting of four random non-specific complementary oligonucleotides to other UHTs. All of the complementary oligonucleotides had a 5′ biotin label and the final concentration in the 50 μl hybridization sample was 10.0 nM, which was the concentration determined from prior experiments to have the maximum level of fluorescent signal with a biotin substrate. Once all of the UHT test samples were read, they were scrutinized based on the highest specific signal obtained, with the signal/noise value being derived from a ratio of the specific signal over the non-specific signal. Cut-off points were determined. An example of a UHT test result is shown in Table 3. Optimization of each individual UHT is readily achieved by UHT testing in these SNP marker assays.

2.3.3. SNP Discrimination

The Luminex system was used with a series of UHT sequences selected to remain single stranded during the assay's course (minimizing hybridization artifacts), and an alternative molecular technique was used to provide the SNP discrimination. This involved attaching marker-specific DNA sequences 3′ to the UHTs used to test for an SNP polymorphism, which were then appropriately labeled for detection. The primary label of interest was biotin as this can be followed with a streptavidin/phycoerythrin conjugate (SA/PE), which, because of its coefficient of extinction, permits the highest amount of sensitivity obtainable in the Luminex system. Direct fluorescent labeling was also tested to compare the potentials offered with that approach, which is simpler to perform due to the fact that the last step of adding a fluorescent conjugate at the end of the Luminex hybridization assay is not required. The final concentration of streptavidin/phycoerythrin conjugate in the Luminex hybridization was approximately 3.3 nM. Therefore, the absolute amount of the biotin substrate in the developing assays could not be significantly more that about ten times that concentration, minimally requiring a wash step before the addition of the SA/PE for high biotin concentrations.

2.4 Short Primer Extension (SPE)

A primer extension method similar to what is used with DNA sequencing was developed. A related method is sometimes called Single Basepair Extension (SBE) or minisequencing, and is a 1 bp extension that incorporates one of two bases found at the SNP site into a single extension primer. Each separate base to be incorporated in the SNP assay has its own fluorescent tag and the extension product is analyzed on a PE Applied Biosystems 377 gel instrument (or similar) to determine which nucleotide analog(s) were incorporated into the extension primer. Basically, the SPE Assay is performed in two steps: (1) extension and labeling using two allele-specific primers (extension primer's 3′ end at SNP position) using as template a PCR product having an SNP site and a nucleotide mixture containing biotin-11-ddATP, and (2) capture of the extension products using a UHT DNA molecular tag onto an oligonucleotide conjugated microsphere followed by data acquisition in the Luminex 100 instrument.

Microfluidics multiplexing of SNP detection involved performing a small length DNA polymerization with two allele-specific primers, while using only one labeled nucleotide for all of the SNP markers. The SNP detection resided with the allele-specific primers with extension only occurring when there was a match between the 3′ end of the allele-specific primer and the SNP site. Since this is more than a 1 bp extension of the primer, but does not completely extend the primer due to the lack of extension time or ddNTP nucleotide inclusion, the method was named the Short Primer Extension Assay or SPE.

2.4.1 Nucleotide Analog Options

The various primer extension protocols that are currently available use Thermosequenase® as the DNA polymerase enzyme in the primer extension assay, however, any enzyme can be used which is capable of polymerizing DNA. The following SPE assay was performed using Thermosequenase®.

There have been many studies examining the incorporation of fluorescently labeled dNTPs and ddNTPs with this enzyme, but very few were directed to biotin-labeled substrates. Therefore, many commercially obtainable biotin analogs were examined side by side with fluorescently labeled nucleotide analogs to see which produced the best signal characteristics.

Table 3 shows the various nucleotide analogs tested for use with the SPE assay. PCR was perfomed as explained in the Microsphere Short Primer Extension Assay (below). PCR products were amplified from homozygous “Fertile” or homozygous “Sterile” genomic DNA samples with PCR pimers from the NVMA marker. The PCR amplicons were then treated with shrimp alkaline phosphatase (SAP) and Exonuclease I to degrade the residual PCR primers and dNTPs. The purified PCR products were then used in separate extension reactions that tested each individual nucleotide analog listed above. After the extension reaction, the extension products were captured with oligonucleotide-tagged microspheres and signal was quantitated using the Luminex 100 instrument.

TABLE 3

| | Allele 1 (fertile) | | Allele 2 (sterile) | |
|---|---|---|---|---|
| Nucleotide Analog | Signal | Background | Signal | Background |

Biotinylated dNTPs

| | | | | |
|---|---|---|---|---|
| Biotin-11-dATP | 10703 | 1153 | 10767 | 665 |
| Biotin-11-dCTP | 7283 | 775 | 6255 | 573 |
| Biotin-16-dUTP | 6562 | 330 | 6860 | 173 |
| Biotin-14-dATP | 3371 | 96 | 3692 | 135 |
| Biotin-4-dCTP | 2508 | 316 | 3407 | 188 |
| Biotin-6-dATP | 2221 | 89 | 2684 | 99 |
| Biotin-14-dCTP | 2038 | 270 | 3519 | 190 |
| Biotin-7-dATP | 969 | 41 | 2282 | 70 |

Fluorescent dNTPs

| | | | | |
|---|---|---|---|---|
| Cy3-dUTP | 171 | 21 | 264 | 54 |
| Cy3-dCTP | 166 | 27 | 367 | 35 |
| TAMRA-6-dCTP | 74 | 30 | 96 | 36 |
| TAMRA-6-dATP | 72 | 32 | 65 | 45 |
| Rhod-4-dUTP | 50 | 18 | 26 | 52 |
| TAMRA-6-dUTP | 41 | 24 | 25 | 29 |

Biotinylated ddNTPs

| | | | | |
|---|---|---|---|---|
| Biotin-11-ddATP | 2188 | 130 | 2722 | 149 |
| Biotin-16-ddUTP | 2059 | 121 | 2887 | 134 |
| Biotin-11-ddCTP | 1964 | 106 | 2621 | 138 |
| Biotin-6-ddATP | 1366 | 73 | 1786 | 105 |

Fluorescent ddNTPs

| | | | | |
|---|---|---|---|---|
| R6G-ddATP | 394 | 18 | 680 | 32 |
| R6G-ddCTP | 153 | 10 | 361 | 27 |
| TAMRA-ddCTP | 125 | 3 | 34 | 20 |
| TAMRA-ddATP | 98 | 5 | 49 | 22 |

2.4.2 Microsphere Short Primer Extension Assay

PCR reactions were prepared using 20 ng of plant genomic DNA in PE Applied Biosystems (Foster City, Calif.) 96 well PCR plates. The PCR reaction mixture included: 300 nM forward and reverse PCR primers, 1×Taq Gold buffer, 0.2 μM dNTPs, 7.5% 10 glycerol, 2.0 MM MgCl₂ and 1.0 Unit Taq Gold (PE Applied Biosystems, Foster City, Calif.) in a 25.0 μl total reaction volume. The samples were amplified in a PE Applied Biosystems 9700 with a 93° C. enzyme activation step followed by 30 cycles of 93° C.-30 s, 60° C.-30 s and 72° C.-30 s. The thermocycling was concluded with a 72° C. step for 5.0 min immediately followed by ramping down to a 4.0° C. hold. After confirmation of a PCR product appearance by agarose gel electrophoresis, 5.0 μl of the PCR product was added to 5.0 μl of a SAP/EXO purification mixture containing 1.0 U of Shrimp Alkaline Phosphatase (USB, Cleveland, Ohio) and 1.0 U of Exonuclease I (USB) in 10.0 mM Tris-Cl, pH, 8.0. The samples were incubated at 37° C. for 45 min followed by a 10-min incubation at 95° C. To these samples, 10 μl of a SPE mixture was added containing 1.0 U Themosequenase® (Amersham Pharmacia Biotech, Piscataway, N.J.), 100.0 μM of each allele-specific extension primer, 1× Themosequenase® buffer, 0.4 μM nucleotide analog (NEN, Roche, Amersham), 2.0 μM dCTP/dGTP/dTTP (PE Applied Biosystems), and 7.5% glycerol. The samples were placed in a 9700 thermocycler and proceeded to a 95° C. incubation for 1.0 min followed by 40 cycles of 95° C.-10 s, and 60° C.-30 s. The denaturation step was then conducted directly in the PCR plate containing the SPE samples since the sample volumes were 20.0 μl. The only modification required for the Luminex procedure was a 1× wash step that was required before the addition of the streptavidin/phycoerythrin conjugate if the biotin-labeled nucleotide analog was used. One hundred μl of 1×TMAC were added to each well and the plate was centrifuged at 3000 rpm for 2.0 min. The supernatant was removed from the microsphere pellet in each well so that 10-20 μl remained. One hundred μl of a 1:800 dilution of streptavidin/phycoeythrin was added to each sample and incubated at 55° C. for at least 5.0 min before reading in the Luminex 100.

In addition to the biotin-11-ddATP nucleotide analog, there are many other nucleotides that can be used to label UHT extension primers. Many of the biotin-conjugated dNTPs have a strong level of signal; e.g. biotin-14-dATP and biotin-6-dATP also show a low non-specific signal. The R6G conjugated ddNTPs also have useful characteristics for the SPE assay. Using these components there is no need for an SA/PE addition step and there is likewise, no washing step required before an SA/PE addition. This can streamline the genotyping procedure. The ddNTP nucleotides were tested at the concentration listed in the assay protocol above, while the dNTP nucleotides were tested at a 10× higher concentration (the unlabeled dNTPs were also 10× more concentrated). Since the incorporation of the dNTP analog does not terminate DNA polymerization, multiple labelings can occur, increasing the sensitivity of the assay. Tetramethylrhodamine (TAMRA), Rhodamine, 3,3,3′,3′ tetramethyl indocarbocyanine (CY3) and rhodamine 6 Green (R6G) were the target fluorescent nucleotides because their emission spectra closely matched the Luminex reporter detection system requirements.

DNA samples with known genotypes for the NVMA marker were obtained. These DNAs and negative PCR controls were amplified with the NVMA-1 and NVMA-2 primers and then treated with SAP/ExoI to prepare them for the Luminex SPE assay. For each nucleotide analog tested, a different UHT was incorporated into the NVMA-3 and NVMA-4 extension primer combination, as were all of the SNP genotype PCR samples. The SPE samples were then read with the Luminex 100 instrument using multiplex data acquisition with two separate microsphere populations. Each population was specific for an allele from the NVMA marker. The results of this experiment are shown in Table 3.

In Table 3, the table is divided into sections consisting of biotin-dNTPs, fluorescent-dNTPs, and biotin-ddNTPs and fluorescent-ddNTPs. This allows easier analysis of the superior nucleotide analogs in each distinctive category to identify which nucleotide analog and SPE assay approach to use in a given situation.

2.4.3 96-Well Plate Format SPE Genotyping

In order to determine how a plate of DNAs with various qualities and concentrations would perform in the same SPE assay, a minimal DNA purification procedure was performed, followed by the genetic analysis protocol. This approach is an efficient way to process very large numbers of samples (hundreds of thousands). A 96-well plate of DNAs that had already been genotyped by the NVMA marker was obtained and the results from the SPE assay compared. Plate #P005 containing genomic DNAs of segregants from an NVMA cross was obtained from Rogers Seeds, Gilroy, Calif. The DNAs on the plate were amplified using the NVMA-1 and NVMA-2 primers, and all of the samples were electrophoresed on an ethidium bromide gel to confirm PCR product formation. All of the samples were then transferred from the PCR plate to a new PCR plate and were SAP/ExoI treated. Next they were tested using the SPE assay and biotin-11-ddATP as the nucleotide analog. A wash step was performed before the SA/PE addition to remove excess un-incorporated biotin labeled ddNTP. The plate was then analyzed in the Luminex 100 using the multiplex data mode, and the genotypes were assigned based on the signals obtained with each specific microsphere. The fluorescent signals were plotted.

When comparing the confirmed genotypes to what was obtained with the Luminex SPE system, there was an exact correlation between the datasets. The only differences that occurred were when the samples showed no signal in the Luminex SPE assay. This was of little concern as it is common to see a small percentage of sample dropout when doing 96-well plate format genotyping. Also, from the graphical representation of the fluorescent signals obtained with each microsphere, it was easy to observed that the genotype calls were easy to perform with each genotype “cluster” being well separated from the others.

When the product of the SPE and ASP assays is single stranded, efficient capture of the corresponding UHT tag on the product occurs with the UHT tag on the bead. With the SPE assay approach, this is easily accomplished because the primer extension reaction does not replicate the reverse strand of the UHTed oligonucleotide. However, the ASP approach can also extend the DNA polymerization occurring in the PCR into the UHTed tag. This can cause weak signals in some assays.

2.5 Allele-Specific PCR (ASP)

The Microsphere Allele-Specific PCR (ASP) assay labels the allele-specific primers used in the allele-specific PCR reaction, and includes the UHT capture onto the Luminex 100 beads. The assay basically has two steps: (1) allele-specific PCR with two allele-specific forward PCR primers and a biotinylated universal reverse PCR primer (allele-specific PCR primers 3′ end at the SNP position), and (2) capture of the PCR products using a UHT DNA molecular tag onto an oligonucleotide-conjugated microsphere followed with analysis in the Luminex 100 instrument.

The nucleotide analogs that were tested included: biotin-7-dATP, biotin-14-dATP, biotin-14-dCTP, biotin-16-dUTP, biotin-6-dATP, biotin-11-dATP, biotin-4-dCTP, biotin-11-dCTP, Cy3-dCTP, Cy3-dUTP, Rhod-4-dUTP, TAMRA-6-dUTP, TAMRA-6-dATP, and TAMRA-6-dCTP. The assay was performed as follows:

2.5.1 Microsphere Allele-Specific PCR Assay

Allele-specific PCR was performed as a three primer PCR reaction. The allele-specific forward primers were used at a 0.15 μM concentration while a universal reverse primer was used at 0.3 μM. Two labeling procedures were tested that included either adding a nucleotide analog to a 10% concentration of each dNTP in the PCR reaction, or the universal reverse primer was 5′ biotin labeled. The remaining components of the PCR reaction included; 20 ng genomic DNA, 1×Taq Gold buffer, 0.2 μM dNTPs, 7.5% glycerol, 2.0 mM MgCl₂ and 1.0 Unit Taq Gold in a 25.0 μl total reaction volume. The samples were amplified in a PE Applied Biosystems 9700 with a 93° C. enzyme activation step followed by 30 cycles of 93° C.-30 sec., 60° C.-30 sec. and 72° C.-30 sec. The thermocycling was concluded with a 72° C. step for 5.0 min immediately followed by ramping down to a 4.0° C. hold. Following the amplification step, PCR products were electrophoresed on a gel to confirm PCR amplification and to determine the correct genotype of the DNA samples. To rid the sample of any allele-specific forward primers, 5.0 μl of the PCR product were treated with 5.0 μl of Exonuclease I solution with the following composition: 10 mM Tris-Cl (pH, 8.0), 1.0 Unit Exonuclease I, and 7.5% glycerol. The samples were incubated at 37° C. for 45 min followed by an enzyme inactivation step at 95° C. for 10 min. To the ExoI purified samples, 10.0 μl of TE buffer (10 mM Tris-Cl, 0.1 mM EDTA, pH, 7.5) was added and the Luminex hybridization assay was immediately performed on them. Since there was a lower concentration of biotin-containing substrate in the initial reaction mixture, a subsequent wash step before streptavidin/phycoerythrin addition was not necessary.

The nucleotide that showed the best signal over background in the Luminex 100 was the biotin-11-dATP, which was approximately double the background amount. It is interesting to note that the biotin attached to the “11” position of the dATP and ddATP nucleotides showed the best results in the ASP and SPE assays, respectively. The PCR products from the biotin-11-dATP labeling showed a significantly slower migration in an EtBr stained gel, suggesting that the labeling did occur.

To optimize the protocol, several standard PCR purification procedures were assayed (Millipore and Qiagen), which demonstrated that using an Exonuclease I treatment was the preferred approach because it had the least cost associated with it and was the easiest to perform. In addition, forward allele-specific PCR primers were re-synthesized with different linkers separating the UHT domain from the marker-specific sequences. The linker prevents the PCR reaction from extending into the UHT domain which ensures that the UHT remains single-stranded and enhances hybridization to the complementary UHT DNA on the microsphere. Also, since Exonuclease I is a 3′ to 5′ exonuclease, the single-stranded UHT sequence is protected from cleavage only if it is extended in the PCR amplification, thus ensuring that the ExoI treatment can still be used. The results from the above experimental conditions are summarized in Table 4B. PCR amplifications for the NVMA marker were set-up using DNAs from each SNP genotype and forward allele-specific PCR primers with various structural characteristics as shown. The PCR products were then examined in a Luminex ASP assay hybridization using two oligonucleotide-conjugated microspheres, each microsphere representing an allele from the SNP site. Table 4A compares the usage of various linkage spacers in two separate experiments while Table 4B shows the benefit of treating the PCR samples with Exonuclease I.

Overall, the results demonstrated that the signal increased 2-3× with ExoI treatment which suggests that approximately one third to one half of the 0.15 μM allele-specific primers were being converted into PCR product. The use of spacer linkers (Oligo Etc. and MWG Biotech, High Point, N.C.) between the UHT and marker specific sequences drastically increased the signal that was obtained (Table 4A). A C12 linker was used having the sequence 5′-CCCCCCCCCCCC-3′ (SEQ ID NO.: 51) and a DS linker also called an abasic spacer. The DS linker is a phosphoramidite without a base, and produces a phosphodiester bond like that in normal DNA but without a nucleotide base associated with it. The C12 and the DS linkers provided the best results when used with the ASP assay. Since the C12 linker had the least cost associated with it, it was chosen for future ASP assays.

Alternative labeling attempts were tested to reduce the expense of performing the ASP procedure. The ideal modification to the assay was to use unlabeled dNTPs with the PCR but to have the reverse PCR primer biotin labeled. The UHTed PCR strand was not directly labeled, but the opposite strand of the PCR product hybridized to it during the UHT capture step and was biotin labeled from the biotinylated PCR primer. The results from this assay demonstrated useful signal levels (Table 4B) and the cost of the labeling component of the assay was reduced by over an order of magnitude.

The ASP assay is useful as an alternative to the SPE assay system. The ASP assay is relatively straightforward to perform, and had the only additional cost of a treatment with ExoI, which is a very inexpensive enzyme.

Another alternative to the assay is the direct phycoerythrin (or other fluorescent label) labeling of DNA to simplify the ASP procedure further. Phycoerythrin is a very large fluorescent molecule (240 kd) and is usually used with a streptavidin conjugate along with the biotin label.

2.5.2 96-Well Plate ASP Genotyping

The reverse biotinylated PCR primer ASP system was used for high-throughput genotyping. A 96-well NVMA plate was used, having DNA samples of various concentration and qualities. Plate #P006 contained genomic DNAs of segregants from a NVMA cross was obtained from Rogers Seeds, Gilroy, Calif. The two forward primers were specific for each allele of the NVMA marker and a biotinylated universal reverse PCR was also used. Following thermocycling, the PCR products were treated with ExoI to remove unused primers. The purified PCR products were evaluated by hybridisation with the respective oligonucleotide-conjugated microspheres, and then quantitated in the Luminex 100 instrument. The results were then plotted and the genotypes assessed.

Upon PCR amplification and gel analysis, a significant percentage of the samples had little or no PCR product present. Allele-specific PCR required a conservative PCR cycle number so that the background values would not increase due to non-specific amplification. Accordingly, the ASP procedure is preferred for PCR samples of relatively high concentration. About 90% of the DNA samples had the capacity to be genotyped correctly, the rest showed no signal. To optimize the ASP assay, the DNA samples used were quantitated and plated out in approximately equal amounts to ensure that an appropriate amount of DNA template was available for the PCR reaction. Also, 1 or 2 additional cycles can be added to the thermocycling program to amplify the low concentration DNA samples without detrimental effects on those samples with a high DNA concentration.

2.5.3 Single SNP Site ASP Assay Evaluation

Results from studies using an individual SNP site to determine genotype with the ASP methodology were analyzed since the most common SNP markers are individual SNPs. Additional ASP primers were designed that could genotype the NVMA marker with only one SNP site. Both SNP sites in the NMVA marker were tested along-side the standard Luminex ASP assay that uses both SNP sites #1 and #2 to generate its SNP data. All of the ASP samples were read on the Luminex 100 instrument and the data were plotted.

2.6 Multiplex ASP and SNP Assay

To analyze whether the present approach could be used for performing genetic analysis on plants at breeding sites, the NVMA marker was multiplexed upon itself by tagging the marker specific sequences with different and unique UHT sets. The samples from each plate were multiplexed by layering the plates on top of each other when aliquoting the samples from the PCR plate to the hybridization plate. DNA plate #3 was replicate plated into three separate 96-well plates and amplified with the NVMA marker. In each separate PCR plate, a combination of unique DNA molecular tags were used so that all 3 plates could be combined into one 96-well plate for the Luminex ASP assay. This was done by adding 5 μl of the PCR sample of the same wells from each of the 3 PCR plates to the respective well of a new 96-well plate. Five microliters of ExoI reagent was added and incubated as usual. In the hybridization step, six microsphere populations were used specific to the DNA molecular tags utilized in the PCR reactions and 3×SA/PE was also added. The 96 samples were then read using the Luminex 100 and graphed.

All datapoints from the NVMA 3× multiplex assay were directly comparable in the “clustering appearance” and in SNP genotypes even though different UHT tags were used with the separate assays. This multiplex approach can be highly useful for laboratories that consistently type the same SNP markers on large DNA sample groups or on replicates of the same DNA samples.

The SPE assay has the advantage that, although more expensive, it is also more robust and has a higher signal level and a higher level of successful SNP genotypes. A major benefit of embodiments of the present invention is the multiplex potential that the system offers. With the reagents used in this assay, it is possible to genotype 3 separate plates and combine them for the Luminex 100 read instead of reading the plates separately. Also, additional NVMA assays can allow even a higher multiplex potential than the 3×SNP genotyping currently available. When the assays are performed in a multiplex fashion, there is the additional advantage of consolidating the use of post-PCR consumables that normally would be used with only a single SNP analysis. When multiplexing, however, the amount of enzymes and other reagents such as SA/PE are typically scaled up to ensure the integrity of the resulting data.

The Luminex 100 instrument has a processivity of up to 20,000 microspheres/sec. The amount of SNPs read with 200 microspheres acquired per SNP could range from 2 to 100/min depending on the level of multiplex with which the assay is designed. Thus, the present invention allows genotyping of up to 40,000 SNPs/day, or more depending on the nature of the assay, the degree of multiplexing, the number of microfluidics readers employed, and the like.

DNA molecular tags can provide a hybridization-based system to capture tagged oligonucleotides out of solution. The UHT DNA sequences as disclosed herein can be conjugated to multiplex microspheres to facilitate the analysis of the captured oligonucleotides in a flow cytometer type of instrument. Two systems, ASP and SPE, are particularly useful. In many cases, the ASP assay is desirable for SNP genotyping due to the ease and low cost of the procedure. In some embodiments, a single SNP assay can be multiplexed upon itself so that 2, 3, 5, or more plates of SNP genotypes can be read with just one plate.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, the invention is intended to embrace all such alternatives, modifications, and variations that fall within the spirit and scope of the following claims.

TABLE 4A

| | DNA Sample in PCR Reaction | | | | | | | |
|---|---|---|---|---|---|---|---|---|
| | Homozygous 1 | | Homozygous 2 | | Homozygous ½ | | No DNA | |
| Linker used | UHT1 | UHT2 | UHT1 | UHT2 | UHT1 | UHT2 | UHT1 | UHT2 |

Experiment 1

| | | | | | | | | |
|---|---|---|---|---|---|---|---|---|
| No linker | 165.13 | 67.88 | 158.8 | 39.19 | 109.7 | 139.6 | 34.8 | 26.9 |
| C9 | 114.5 | 18.6 | 41.51 | 163.8 | 98.3 | 61.3 | 40.5 | 20.7 |
| C12 | 716.5 | 44.7 | 49.6 | 716.7 | 367.7 | 209.4 | 32.5 | 20.3 |
| C18 | 314.0 | 39.7 | 56.7 | 240.0 | 150.3 | 135.7 | 31.4 | 24 |

Experiment 2

| | | | | | | | | |
|---|---|---|---|---|---|---|---|---|
| C12 | 601.4 | 45.5 | 58.0 | 751.1 | 440.8 | 499.3 | 48.0 | 38.9 |
| D spacer | 556.6 | 52.1 | 58.2 | 688.1 | 489.7 | 475.6 | 64.4 | 40.5 |

TABLE 4B

| | DNA Sample in PCR Reaction | | | | | | | |
|---|---|---|---|---|---|---|---|---|
| | Homozygous 1 | | Homozygous 2 | | Homozygous ½ | | No DNA | |
| Experimental Parameters | UHT1 | UHT2 | UHT1 | UHT2 | UHT1 | UHT2 | UHT1 | UHT2 |

Biotin Incorporated PCR

| | | | | | | | | |
|---|---|---|---|---|---|---|---|---|
| No linker, no ExoI | 195.5 | 50.03 | 64.4 | 592.7 | 156 | 367.2 | 49.46 | 29.2 |
| No linker, plus ExoI | 751.9 | 266.1 | 157.8 | 1613.1 | 706.3 | 1224 | 65.9 | 29.7 |
| C12, no ExoI | 1980.3 | 685.8 | 64.6 | 933.1 | 761.4 | 431.7 | 64.5 | 24.3 |
| C12, plus ExoI | 4474.8 | 354 | 2662.5 | 2311.2 | 3342.7 | 2417.5 | 49.2 | 24.4 |

Biotinylated Reverse PCR Primer

| | | | | | | | | |
|---|---|---|---|---|---|---|---|---|
| No linker, no ExoI | 53.8 | 23.3 | 37.3 | 52 | 50.5 | 47.5 | N/A | N/A |
| No linker, plus ExoI | 102.3 | 40.9 | 43.1 | 179.1 | 116.3 | 144.1 | 33.6 | 18.5 |
| C12, no ExoI | 255.1 | 26.2 | 44.6 | 278.9 | 176.7 | 147.8 | 46 | 25.3 |
| C12, plus ExoI | 519.6 | 41.9 | 45.4 | 652.2 | 449.3 | 448.1 | 46.2 | 24.3 |

What is claimed is:

1. A method for determining polynucleotide expression comprising:
a) providing at least one target polynucleotide, said polynucleotide having a 3′ end and a 5′ end;
b) providing a first oligonucleotide primer, wherein a portion of said first primer is capable of hybridizing to said target polynucleotide;
c) obtaining a first strand cDNA by reverse transcription of said target polynucleotide, said first strand cDNA having a 5′ end and a 3′ end, wherein said 5′ end of said first strand cDNA contains a sequence corresponding to said first oligonucleotide primer and said 3′ end of said first strand cDNA comprises at least one nucleotide that extends beyond the 5′ end of said target polynucleotide to provide a single-stranded extension;
d) providing a second oligonucleotide primer, wherein at least a portion of said second oligonucleotide primer is capable of hybridizing to said single-stranded extension;
e) extending said first strand cDNA using said second oligonucleotide primer as a template to produce an extended first strand cDNA containing said first oligonucleotide primer and a region complementary to said second oligonucleotide primer;
f) amplifying said extended first strand cDNA in the presence of at least one detectable label to produce amplified cDNA such that said amplified cDNA contains said at least one detectable label;
g) digesting said amplified cDNA to produce a digested cDNA;
g) hybridizing said digested cDNA to a capture probe coupled to a solid particle under stringent conditions wherein said capture probe is specific for said target polynucleotide and said particle identifies said capture probe; and
h) determining if said digested cDNA has hybridized to said capture probe thereby identifying said target polynucleotide.

2. The method of claim 1, wherein said particle comprises a fluorescent particle.

3. The method of claim 2, wherein said fluorescent particle comprises a fluorescent microbead or microsphere.

4. The method of claim 2, wherein said fluorescent particle comprises a plurality of groups of fluorescent particles, the particles of each group having a unique fluorescent signature and comprising a capture probe specific for a single target polynucleotide.

5. The method of claim 4, wherein said fluorescent particles comprise microbeads or micro spheres.

6. The method of claim 5, wherein determining if said digested cDNA has hybridized to said capture probe thereby identifying said target polynucleotide is accomplished by flow cytometry.

7. The method of claim 6, wherein multiple capture probes hybridize to the same target polynucleotide at different locations on said target polynucleotide.

8. The method of claim 7, wherein said first oligonucleotide primer comprises the sequence n_{y}(t)ₓvn, where x is an integer between 4 and 50 and y is an integer between 10 and 50.

9. The method of claim 8, wherein said second oligonucleotide primer comprises the sequence n_{y}(g)ₓ, where y is an integer between 10 and 50 and x is an integer between 1 and 6.

10. The method of claim 9 wherein said first oligonucleotide primer and said second oligonucleotide primer contain a restriction site.

11. A method for diagnosing a disease, condition, disorder or predisposition in a test subject comprising, determining polynucleotide expression in a test subject by the method of claim 1; determining polynucleotide expression in a reference subject known to have said disease, condition, disorder, or predisposition by the method of claim 1; and comparing polynucleotide expression in said test subject to polynucleotide expression in said reference subject.

12. A method for determining the physiological or developmental state of a cell or tissue comprising, determining polynucleotide expression in a test cell or tissue by the method of claim 1; determining polynucleotide expression in a reference cell or tissue of a known physiological or developmental state by the method of claim 1; and comparing polynucleotide expression in said test cell or tissue to polynucleotide expression in said reference cell or tissue.

13. A method for detecting a single nucleotide polymorphism comprising:
a) providing at least one primer pair, said primer pair containing a reverse primer and a forward primer comprising a 3′ end specific for an allele of a single nucleotide polymorphism of interest and a hybridization tag that identifies the primer, said hybridization tag not complementary to the sequence containing said single nucleotide polymorphism of interest;
b) combining said at least one primer pair with a sample containing single-stranded polynucleotides under stringent conditions which allow hybridization of said primers to complementary sequences in said single-stranded polynucleotides;
c) extending hybridized primers by primer extension to produce an extension product wherein said extension product comprising said hybridization tag and a detectable label;
d) hybridizing said extension products by said hybridization tag or the complement thereof under stringent conditions to a capture probe wherein said capture probe is coupled to a particle, said particle identifying said capture probe;
e) detecting the hybridizaton of said extension product to said capture probe by the presence of said detectable label; and
f) determining the identity of said single nucleotide polymorphism based on the identity of said particle.

14. The method of claim 13, wherein said reverse primer comprises said detectable label.

15. The method of claim 14, wherein said reverse primer pair is a universal reverse primer.

16. The method of claim 13, wherein c) is repeated at least once.

17. The method of claim 13, wherein said at least one primer pair comprises a plurality of primer pairs specific for a plurality of single nucleotide polymorphisms.

18. The method of claim 13, wherein said detection is by flow cytometry.

19. A method for diagnosing a disease, condition, disorder or predisposition in a subject comprising, obtaining a biological sample containing at least one polynucleotide from said subject and analyzing said at least one polynucleotide to detect the presence or absence of a single nucleotide polymorphism by the method of claim 13, wherein said single nucleotide polymorphism is associated with a disease, condition, disorder or predisposition.

20. A method for detecting a single nucleotide polymorphism comprising:
a) providing at least one oligonucleotide primer comprising a hybridization tag that identifies said primer, said primer having a 3′ end specific for a single nucleotide polymorphism of interest;
b) combining said at least one primer with a sample containing single-stranded polynucleotides under stringent conditions which allow hybridization of said primer to complementary sequences in said single-stranded polynucleotides;
c) extending hybridized primers by primer extension to produce an extension product, said extension product comprising said hybridization tag and a detectable label;
d) hybridizing said extension product by said hybridization tag under stringent conditions to a capture probe, said capture probe couple to a particle that identifies said capture probe;
e) detecting the hybridization of said extension product to said capture probe using said detectable label; and
f) determining the identity of said single nucleotide polymorphism based on the identity of said particle.

21. The method of claim 20, wherein said at least one primer comprises a plurality of primers each specific for a different single nucleotide polymorphism.

22. The method of claim 20, wherein said at least one primer comprises a group of at least 2 primers, each primer in said group having a 3′ end specific for a different allele of a single nucleotide polymorphism of interest.

23. The method of claim 22 further comprising a plurality of said primer groups, each primer group specific for a different single nucleotide polymorphism of interest.

24. The method of claim 20, wherein the 3′ end of said primer is immediately adjacent to location of the single nucleotide polymorphism of interest.

25. The method of claim 24, wherein said primer extension is a single base primer extension.

26. The method of claim 25, wherein said single base extension is achieved by using only a single type of nucleoside triphosphate.

27. The method of claim 25, wherein said single base extension is achieved by using at least one-chain terminating nucleoside triphosphate.

28. The method of claim 27, wherein said chain-terminating nucleotide-triphosphate is a dideoxynucleoside triphosphate.

29. The method of claim 25, wherein said single base extension is achieved by using a plurality of chain-terminating nucleoside triphosphates, each comprising a unique label.

30. The method of claim 29, wherein said chain-terminating nucleotide triphosphates are dideoxynucleoside triphosphates.

31. A method for diagnosing a disease, condition, disorder or predisposition in a subject comprising, obtaining a biological sample containing at least one polynucleotide from said subject and analyzing said at least one polynucleotide to detect the presence or absence of a single nucleotide polymorphism by the method of claim 20, wherein said single nucleotide polymorphism is associated with a disease, condition, disorder or predisposition.

32. A method for selecting hybridization tags comprising identifying non-coding sequences of between about 10 to about 30 nucleotides long, wherein said sequences lack hairpin structures and duplex-forming abilities; identifying those sequences having a GC content of between about 40% to about 50% and a Tₘ that varies by no more than 2° C.; and selecting such sequences as hybridization tags.

33. A hybridization tag produced by the method of claim 32.

34. A universal hybridization tag comprising a nucleotide sequence selected from the group consisting of SEQ ID NOS 3, 4, 5, 6, 9, 12, 13, 14, 15, 16, 17, 18, 19, 20, 21, 22, 24, 25, 26, 28, 29, 30, 31, 32, 36, 38, 40, 41, 42, 43, and 45.

35. A universal hybridization tag consisting of a nucleotide sequence selected from the group consisting of SEQ ID NOS 3, 4, 5, 6, 9, 12, 13, 14, 15, 16, 17, 18, 19, 20, 21, 22, 24, 25, 26, 28, 29, 30, 31, 32, 36, 38, 40, 41, 42, 43, and 45.
